(19) United States
(12) Patent Application Publication
(10) Pub. No.: US 2025/0223262 A1
(43) Pub. Date: Jul. 10, 2025

(54) SYNTHESIS OF MORPHINANS WITH REDUCED HERG ACTIVITY AND MOP BINDING

(71) Applicants: Heinrich-Heine-Universität Düsseldorf, Düsseldorf (DE); DEUTSCHE DIABETES-FORSCHUNGSGESELLSCHAFT E.V., Düsseldorf (DE); TAROS CHEMICALS GMBH & CO. KG, Dortmund (DE)

(72) Inventors: Eckhard LAMMERT, Düsseldorf (DE); Okka SCHOLZ, Köln (DE); Silke OTTER, Duisburg (DE); Diran HEREBIAN, Düsseldorf (DE); Torsten HOFFMANN, Dortmund (DE); Miguel Angel SANZ, Dortmund (DE); Laurenz KRAMP, Dortmund (DE); Laura Mariana LEVY, Dortmund (DE); Stanimira HRISTEVA, Dortmund (DE)

(21) Appl. No.: 18/851,265

(22) PCT Filed: Mar. 30, 2023
(86) PCT No.: PCT/EP2023/058373

§ 371 Date: Sep. 26, 2024

(30) Foreign Application Priority Data
Mar. 31, 2022 (EP) 22165895.8

(51) Int. Cl. C07D 221/28; A61K 31/485; A61P 3/10; C07D 401/04; C07D 403/12

(52) U.S. Cl. CPC C07D 221/28; A61K 31/485; A61P 3/10; C07D 401/04; C07D 403/12

(57) ABSTRACT

The invention relates to morphinan-derivatives, processes for their preparation, medicaments containing them and the use of these morphinan-derivatives for the preparation of medicaments.

### CLAIM OF PRIORITY

This application is a national phase filing under 35 USC § 371 fron PCT (International) Patent Application Serial Number PCT/EP2023/058373 filed on Mar. 30, 2023 and claims priority therefrom. This application and PCT/EP2023/05873 further claim priority to European patent Application Number 22 165 895.8 filed on Mar. 31, 2022. PCT Patent Application Serial Number PCT/EP2023/058373 and EP Patent Application Number EP 22 165 895.8 are each incorporated herein by reference in its entirety.

### FIELD

The invention relates to morphinan-derivatives, processes for their preparation, medicaments containing them and the use of these morphinan-derivatives for the preparation of medicaments.

### BACKGROUND

Morphinan-derivatives and in particular dextromethorphan-derivatives are particularly useful to suppress coughing by blocking NMDA receptors in the central nervous system (CNS). However, besides their activity in the central nervous system, their activities in peripheral tissue has attracted attention in clinical research over the last years.

WO 2013/029762 discloses that dextromethorphan and other morphinan-derivatives target NMDA receptors on pancreatic islets for use in the treatment of insulin-dependent diabetes mellitus, non-insulin-dependent diabetes mellitus, obesity, and/or diabetic nephropathy (see Marquard et al., Nat. Med. 2015; 21:363-372; see also Ashcroft et al., Cell 2012; 148: 1160-1171). Dextromethorphan has also been successfully used in the treatment of neuropathic pain, such as diabetic neuropathy (see Zhou et al., Expert Rev Clin Pharmacol 2011; 4: 379-388; Shaibani et al., Pain Med 2012; 13: 243-254). Further, preclinical studies revealed that NMDA receptor antagonists may inhibit metastasis and tumor growth and an elevated coexpression of NMDA receptors and glutamate exporters in cancer cells correlates with a poor prognosis for cancer patients (see Li et al., Cell 2013; 153: 86-100).

Moreover, inhibition of NMDA receptors reduces ischemia-reperfusion (I/R) injury in different cells and organs, including heart, kidney and sciatic nerve (see Liu et al., J. Cardiovasc. Pharmacol. 2017; 70: 329-338; Yang et al., Am. J. Physiol. Renal Physiol. 2008; 294: F1433-F1440; Ke et al., Exp. Therapeut. Med. 2016; 11: 1563-1572). NMDA receptor blockage has also been shown to reduce cell death induced by “excitotoxicity”, which develops in stroke and neurodegenerative human diseases (Wang & Swanson, Front. Neurosci. 2020; 14: 861; Szabo et al., Nat. Rev. Drug Disc. 2007; 6: 662-680).

For targeting peripheral tissues, such as pancreatic islets and other ischemic or excitotoxic tissues, it would be desirable to suppress the central activity of dextromethorphan in order to reduce the frequency and intensity of central nervous adverse effects as observed upon administration of elevated doses of dextromethorphan (see Marquard et al., Nat. Med. 2015; 21:363-372; Scholz et al., The NMDA receptors 2017; 121-134). The occurrence of central nervous adverse effects, the risk of a so far incalculable long-term neurotoxicity, as well as a potential development of dependency, which may occur in treatment with conventional morphinans or NMDA receptor antagonists, make it problematic to use them at higher concentrations over an extended treatment period (see Logan et al., J Anal Toxicol 2009; 33: 99-103; Olney et al., Science 1989; 244: 1360-2; Zhou et al., Expert Rev Clin Pharmacol 2011; 4: 379-388).

WO 2011/014003 A2 relates to (+)-3-hydroxymorphinan derivatives and a pharmaceutical composition comprising the same as an active ingredient.

J. A. Fernández-Salas et al., Chem Commun 52(83) 2016, 12364-12367 relates to metal-free CH thioarylation or arenes using sulfoxides: a direct, general diaryl sulfide synthesis.

D. Wang et al., Angew Chem Int Ed. 59(37) 2020, 15918-15922 relates to trifluoromethyl sulfoxides as reagents for metal-free C—H trifluoromethylation.

WO 2017/093519 A1 relates to dextrorphan-derivatives, pharmaceutical compositions and pharmaceutical dosage forms containing such dextrorphan-derivatives as well as the use of those dextrorphan-derivatives and/or compositions for treating and preventing diseases and conditions in man and mammals.

### SUMMARY

There is a demand for morphinans that overcome the drawbacks of the prior art. It is therefore an object of the invention to provide compounds that have advantages compared to the prior art.

This object has been achieved by the subject-matter of the patent claims.

It has been surprisingly found that the compounds according to the invention increase glucose-stimulated insulin secretion and survival of pancreatic islets, while passing the blood-brain barrier to a significantly lower extent than dextromethorphan. The compounds according to the invention are capable of decreasing blood glucose concentrations in mice after intraperitoneal or oral administration. Furthermore, application of the compounds according to the invention results in fewer behavioral changes and they are less toxic than dextromethorphan. In particular, it has been surprisingly found that the compounds according to the invention show significantly reduced binding of hERG (potassium voltage-gated channel subfamily H member 2) and -type opioid (MOP) receptor compared to dextromethorphan or its derivative Lam39M, which has fewer central nervous side effects (Scholz et al., Cell Chem. Biol. 2021; 28: 1474-1488). Further, it is possible to chemically synthesize and purify the compounds according to the invention.

A first aspect of the invention relates to a compound according to general formula (I)

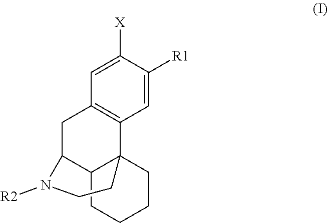

wherein
R1 means —H, —OH, —CO₂H, —R0, —OR0, —OC(═O)R0, —OC(═O)OR0 or —OC(═O)NHR0; preferably —OR0; more preferably —OC₁₋₆-alkyl, unsubstituted or substituted; still more preferably —OCH₃;
R2 means —H, —R0, —C(═O)R0, —C(═O)OR0 or —C(═O)NHR0; preferably —R0; more preferably -C₁₋₆-alkyl, unsubstituted or substituted; still more preferably —CH₃;
X means —SR0 or —NR3R4, wherein
R3 means —H, —R0, —C(═O)R0, —C(═O)OR0, —C(═O)NHR0, —C(═O)N(R0)₂, —C(═O)NH₂, —C(═O)CH(NH₂)R0 or —S(═O)₂R0; preferably —H or —R0; more preferably —H or -C₁₋₆-alkyl, unsubstituted or substituted; still more preferably —H or —CH₃; and R4 means —C(═O)R0, —C(═O)OR0, —C(═O)NHR0, —C(═O)N(R0)₂, —C(═O)NH₂, —C(═O)CH(NH₂)R0 or —S(═O)₂R0; preferably —C(═O)R0 or —C(═O)NHR0; more preferably —C(═O)R0 or —C(═O)NH—C₁₋₆-alkyl, unsubstituted or substituted; still more preferably —C(═O)R0 or —C(═O)NH—CH₃;
or R3 and R4 together with the nitrogen atom to which they are attached form an azetidine ring, a pyrrolidine ring, a piperidine ring, a piperazine ring, a pyrazole ring, a triazole ring, an imidazole ring, or a spirocyclic compound/moiety containing an azetidine ring and an oxetanyl ring, in each case independently unsubstituted or substituted with one or two substituents independently selected from the group consisting of ═O, —F, —CH₃, —C(═O)NH₂, —C(═O)NHCH₃, —SO₂CH₃, —NH₂ and —N(CH₃)₂;
wherein R0 in each case independently means -C₁₋₆-alkyl, -C₃₋₁₂-cycloalkyl, -C₁₋₆-alkylene-C₃₋₁₂-cycloalkyl, -C₃₋₁₂-heterocycloalkyl, -C₁₋₆-alkylene-C₃₋₁₂-heterocycloalkyl, -C₆₋₁₄-aryl, -C₁₋₆-alkylene-C₃₋₆-aryl, -C₃₋₁₄-heteroaryl or -C₁₋₆-alkylene-C₃₋₁₄-heteroaryl, in each case independently unsubstituted or substituted;
wherein in each case -C₁₋₆-alkyl may be linear or branched, saturated or unsaturated, unsubstituted or substituted;
wherein in each case -C₁₋₆-alkylene- may be linear or branched, saturated or unsaturated, unsubstituted or substituted;
wherein in each case -C₃₋₁₂-cycloalkyl may be saturated or unsaturated, unsubstituted or substituted;
wherein in each case -C₃₋₁₂-heterocycloalkyl may be may be saturated or unsaturated, unsubstituted or substituted;
wherein in each case -C₆₋₁₄-aryl may be unsubstituted or substituted;
wherein in each case -C₃₋₁₄-heteroaryl may be unsubstituted or substituted;
wherein in each case, and unless expressly defined otherwise, “substituted” means mono- or polysubstituted with one or more substituents independently of one another selected from -C₁₋₆-alkyl, —F, —Cl, —Br, —I, and —NH₂;
or a physiologically acceptable salt thereof.

### BRIEF DESCRIPTION OF THE DRAWINGS

FIG. 1 shows experimental data demonstrating that compound 7 (5) (Kom56) increases the glucose-stimulated insulin secretion from mouse pancreatic islets.

FIG. 2 shows experimental data demonstrating that compound 7 (5) (Kom56) increases plasma insulin concentrations and improves glucose tolerance after intraperitoneal administration.

FIG. 3 shows experimental data demonstrating that compound 7 (5) (Kom56) increases plasma insulin concentrations and improves glucose tolerance after peroral administration.

FIG. 4 shows experimental data demonstrating that compound 7 (5) (Kom56) displays a reduced blood-brain barrier (BBB) permeability.

FIG. 5 shows experimental data demonstrating that compound 7 (5) (Kom56) reduces touch sensitivity during mechanical stimulation with Von Frey monofilaments.

FIG. 6 shows experimental data demonstrating that compound 7 (5) (Kom56) protects mouse pancreatic islets from cell death induced by oxidative stress.

FIG. 7 shows experimental data demonstrating that compound 7 (5) (Kom56) protects mouse pancreatic islets from streptozotocin-induced cell death.

FIG. 8 shows a histogram for compound 7 (5) (Kom56) with regard to % inhibition of control specific binding (test concentration 1.0 10⁻⁵ M).

FIG. 9 shows a histogram for dextromethorphan (DXM) (1) with regard to % inhibition of control specific binding (test concentration 1.0 10⁻⁵ M).

FIG. 10 shows a histogram for (9S, 13S, 14S)-2-(1H-Imidazol-1-yl)-3-methoxy-17-methyl morphinan dihydrobromide (DXM-2) with regard to % inhibition of control specific binding (test concentration 1.0 10⁻⁵ M).

### DETAILED DESCRIPTION

According to the invention, unless expressly stated otherwise, “—C₁₋₄-alkyl”, “—C₁₋₆-alkyl” and any other alkyl residues can be linear or branched, saturated or unsaturated. Linear saturated alkyl includes methyl, ethyl, n-propyl, n-butyl, n-pentyl and n-hexyl. Examples of branched saturated alkyl include but are not limited to iso-propyl, sec-butyl, and tert-butyl. Examples of linear unsaturated alkyl include but are not limited to vinyl, propenyl, allyl, and propargyl.

According to the invention, unless expressly stated otherwise, “—C₁₋₄-alkyl”, “—C₁₋₆-alkyl” and any other alkyl residues can be unsubstituted, mono- or polysubstituted. Examples of substituted alkyl include but are not limited to —CH₂CH₂OH, —CH₂CH₂OCH₃, —CH₂CH₂CH₂OCH₃, —CH₂CH₂S(═O)₂CH₃, —CH₂C(═O)NH₂, —C(CH₃)₂C(═O)NH₂, —CH₂C(CH₃)₂C(═O)NH₂, —CH₂CH₂C(═O)N(CH₃)₂, —C(═O)CH₃, —C(═O)C(CH₃)₃, —C(═O)—CH₂—CF₃, —C(═O)N(CH₃)₂, —C(═O)NH₂, and —S(═O)₂—CH₃.

According to the invention, unless expressly stated otherwise, “—C₁₋₆-alkylene-”, “—C₁₋₄-alkylene” (preferred form of “-C₁₋₆-alkylene-”) and any other alkylene residue can be unsubstituted, monoor polysubstituted. Examples of saturated alkylene include but are not limited to —CH₂—, —CH(CH₃)—, —C(CH₃)₂—, —CH₂CH₂—, —CH(CH₃)CH₂—, —CH₂CH(CH₃)—, —CH(CH₃)—CH(CH₃)—, —C(CH₃)₂CH₂—, —CH₂C(CH₃)₂—, —CH(CH₃)C(CH₃)₂—, —C(CH₃)₂CH(CH₃)—, C(CH₃)₂C(CH₃)₂—, —CH₂CH₂CH₂—, and —C(CH₃)₂CH₂CH₂—. Examples of unsaturated alkylene include but are not limited to —CH═CH—, —C≡C—, —C(CH₃)═CH—, —CH═C(CH₃)—, —C(CH₃)═C(CH₃)—, —CH₂CH═CH—, —CH═CHCH₂—, —CH═CH—CH═CH—, and —CH═CH—C≡C—.

According to the invention, unless expressly stated otherwise, “—C₁₋₆-alkylene-”, “—C₁₋₄-alkylene” and any other alkylene residue can be unsubstituted, mono- or polysubstituted. Examples of substituted —C₁₋₆-alkylene- include but are not limited to —CHF—, —CF₂—, —CHOH— —C(═O)—, and —C(═O)CH(NH₂)—.

According to the invention, moieties may be connected through -C₁₋₆-alkylene-, i.e. the moieties may not be directly bound to the core structure of compound according to general formula (I), but may be connected to the core structure of compound according to general formula (I) or its periphery through a -C₁₋₆-alkylene- linker.

According to the invention, “—C₃₋₁₂-cycloalkyl” means a non-aromatic, monocyclic, bicyclic or tricyclic moiety comprising 3 to 12 ring carbon atoms but no heteroatoms in the ring. Examples of preferred saturated -C₃₋₁₂-cycloalkyl moieties according to the invention include but are not limited to cyclopropane, cyclobutane, cyclopentane, cyclohexane, cycloheptane, cyclooctane, hydrindane, and decaline. Examples of preferred unsaturated -C₃₋₁₂-cycloalkyl according to the invention include but are not limited to cyclopropene, cyclobutene, cyclopentene, cyclopentadiene, cyclohexene, 1,3-cyclohexadiene, and 1,4-cyclohexadiene. The -C₃₋₁₂-cycloalkyl, which is bonded to the compound according to the invention, in its periphery may optionally be condensed with a -C₃₋₁₂-heterocycloalkyl, saturated or unsaturated, unsubstituted, mono- or polysubstituted; and/or with a -C₆₋₁₄-aryl, unsubstituted, mono- or polysubstituted; and/or with a -C₃₋₁₄-heteroaryl, unsubstituted, mono- or polysubstituted. Under these circumstances, the ring atoms of the condensed moieties are not included in the 3 to 12 ring atoms of the -C₃₋₁₂-cycloalkyl. Examples of -C₃₋₁₂-cycloalkyl moieties condensed with -C₃₋₁₂-heterocycloalkyl moieties include but are not limited to octahydro-1H-indol, decahydroquinoline, decahydroisoquinoline, octahydro-2H-benzo[b][1,4]oxazin, and decahydroquinoxalin, which in each case are connected through the -C₃₋₁₂-cycloalkyl. Examples of -C₃₋₁₂-cycloalkyl moieties condensed with -C₆₋₁₄-aryl moieties include but are not limited to 2,3-dihydro-1H-indene and tetraline, which in each case are connected through the -C₃₋₁₂-cycloalkyl. Examples of -C₃₋₁₂-cycloalkyl moieties condensed with -C₃₋₁₄-heteroaryl moieties include but are not limited to 5,6,7,8-tetrahydroquinoline and 5,6,7,8-tetrahydroquinazoline, which in each case are connected through the -C₃₋₁₂-cycloalkyl.

According to the invention, the -C₃₋₁₂-cycloalkyl may optionally be connected through -C₁₋₆ alkylene-, i.e. the -C₃₋₁₂-cycloalkyl may not be directly bound to the compound according to general formula (I) but may be connected thereto through a -C₁₋₆-alkylene- linker. Examples include but are not limited to —CH₂-cyclopropyl, —CH₂-cyclobutyl, —CH₂-cyclopentyl, —CH₂-cyclohexyl, —CH₂CH₂-cyclopropyl, —CH₂CH₂-cyclobutyl, —CH₂CH₂-cyclopentyl, —CH₂CH₂-cyclohexyl, and —C(═O)CH(NH₂)—(C₃Hscycloalkyl).

According to the invention, unless expressly stated otherwise, the -C₃₋₁₂-cycloalkyl can be unsubstituted, mono- or polysubstituted. Examples of substituted -C₃₋₁₂-cycloalkyl moieties include but are not limited to —CH₂-1-hydroxy-cyclobutyl.

According to the invention, “—C₃₋₁₂-heterocycloalkyl” means a non-aromatic, monocyclic, bicyclic or tricyclic moiety comprising 3 to 12 ring atoms, wherein each cycle comprises independently of one another 1, 2, 3, 4 or more heteroatoms independently of one another selected from the group consisting of nitrogen, oxygen and sulfur, whereas sulfur may be oxidized (S(═O) or (S(═O)₂), whereas the remaining ring atoms are carbon atoms, and whereas bicyclic or tricyclic systems may share common heteroatom(s). Examples of preferred saturated -C₃₋₁₂-heterocycloalkyl moieties according to the invention include but are not limited to aziridin, azetidine, pyrrolidine, imidazolidine, pyrazolidine, piperidine, piperazine, triazolidine, tetrazolidine, oxiran, oxetane, tetrahydrofuran, tetrahydropyran, thiirane, thietane, tetrahydrothiophene, diazepane, oxazolidine, isoxazolidine, thiazolidine, isothiazolidine, thiadiazolidine, morpholine, thiomorpholine. Examples of preferred unsaturated -C₃₋₁₂-heterocycloalkyl moieties according to the invention include but are not limited to oxazoline, pyrazoline, imidazoline, isoxazoline, thiazoline, isothiazoline, and dihydropyran. The -C₃₋₁₂-heterocycloalkyl, which is bonded to the compound according to the invention, in its periphery may optionally be condensed with a -C₃₁₂-cycloalkyl, saturated or unsaturated, unsubstituted, mono- or polysubstituted; and/or with a -C₆₋₁₄-aryl, unsubstituted, mono- or polysubstituted; and/or with a -C₃₋₁₄-heteroaryl, unsubstituted, mono- or polysubstituted. Under these circumstances, the ring atoms of the condensed moieties are not included in the 3 to 12 ring atoms of the -C₃₋₁₂-heterocycloalkyl moieties. Examples of -C₃₋₁₂-heterocycloalkyl moieties condensed with -C₃₋₁₂-cycloalkyl moieties include but are not limited to octahydro-1H-indol, decahydroquinoline, decahydroisoquinoline, octahydro-2H-benzo[b][1,4]oxazin, and decahydroquinoxalin, which in each case are connected through the -C₃₋₁₂-heterocycloalkyl. An examples of a -C₃₋₁₂-heterocycloalkyl condensed with a -C₆₋₁₄-aryl includes but is not limited to 1,2,3,4-tetrahydroquinoline, which is connected through the -C₃₋₁₂-heterocycloalkyl. An example of a -C₃₋₁₂-heterocycloalkyl condensed with a -C₃₋₁₄-heteroaryl moieties includes but is not limited to 5,6,7,8-tetrahydro-[1,2,4]triazolo[1,5-a]pyrazine, which is connected through the -C₃₋₁₂-heterocycloalkyl.

According to the invention, the -C₃₋₁₂-heterocycloalkyl may optionally be connected through -C₁₋₆-alkylene-, i.e. the -C₃₋₁₂-heterocycloalkyl may not be directly bound to the compound according to general formula (I) but may be connected thereto through a -C₁₋₆-alkylene- linker. Said linker may be connected to a carbon ring atom or to a hetero ring atom of the -C₃₋₁₂-heterocycloalkyl. Examples include but are not limited to —CH₂-oxetane, —CH₂-pyrrolidine, —CH₂-piperidine, —CH₂-morpholine, —CH₂CH₂-oxetane, —CH₂CH₂-pyrrolidine, —CH₂CH₂-piperidine, and —CH₂CH₂-morpholine.

According to the invention, unless expressly stated otherwise, the -C₃₋₁₂-heterocycloalkyl can be unsubstituted, mono- or polysubstituted. Examples of substituted -C₃₋₁₂-heterocycloalkyl moieties include but are not limited to 1-methyl-5-pyrrolidinyl, 3-fluoro-5-pyrrolidinyl, 1-methyl-5-piperazinyl, 2-carboxamido-N-pyrrolidinyl-, 3,4-dihydroxy-N-pyrrolidinyl, 3-hydroxy-N-pyrimidinyl, 3,4-dihydroxy-N-pyrimidinyl, 3-oxo-N-piperazinyl, -tetrahydro-2H-thiopyranyl dioxide and thiomorpholinyl dioxide.

According to the invention, “—C₆₋₁₄-aryl” means an aromatic, monocyclic, bicyclic or tricyclic moiety comprising 6 to 14 ring carbon atoms but no heteroatoms in the ring. Examples of preferred -C₆₋₁₄-aryl moieties according to the invention include but are not limited to benzene, naphthalene, anthracen, and phenanthren. The -C₆₋₁₄-aryl, which is bonded to the compound according to the invention, in its periphery may optionally be condensed with a -C₃₋₁₂-cycloalkyl, saturated or unsaturated, unsubstituted, mono- or polysubstituted; and/or with a -C₃₋₁₂-heterocycloalkyl, saturated or unsaturated, unsubstituted, mono- or polysubstituted; and/or with a -C₃₋₁₄-heteroaryl, unsubstituted, mono- or polysubstituted. Under these circumstances, the ring atoms of the condensed moieties are not included in the 6 to 14 ring carbon atoms of the 6-14-membered heterocycloalkyl moieties. Examples of -C₆₋₁₄-aryl moieties condensed with -C₃₋₁₂-cycloalkyl moieties include but are not limited to 2,3-dihydro-1H-indene and tetraline, which in each case are connected through the -C₆₋₁₄-aryl. An example of a -C₆₋₁₄-aryl condensed with a -C₃₋₁₂-heterocycloalkyl includes but is not limited to 1,2,3,4-tetrahydroquinoline, which is connected through the -C₆₋₁₄-aryl. Examples of -C₆₋₁₄-aryl moieties condensed with -C₃₋₁₄-heteroaryl moieties include but are not limited to quinoline, isoquinoline, phenazine and phenoxacine, which in each case are connected through the -C₆₋₁₄-aryl.

According to the invention, the -C₆₋₁₄-aryl may optionally be connected through -C₁₋₆-alkylene, i.e. the -C₆₋₁₄-aryl may not be directly bound to the compound according to general formula (I) but may be connected thereto through a -C₁₋₆-alkylene- linker. Said linker may be connected to a carbon ring atom or to a hetero ring atom of the -C₆₋₁₄-aryl. Examples include but are not limited to —CH₂—C₆H₅, —CH₂CH₂—C₆H₅ and —CH═CH—C₆H₅.

According to the invention, unless expressly stated otherwise, the -C₆₋₁₄-aryl can be unsubstituted, mono- or polysubstituted. Examples of substituted -C₆₋₁₄-aryl moieties include but are not limited to 2-fluorophenyl, 3-fluorophenyl, 2-methoxyphenyl and 3-methoxyphenyl.

According to the invention, “—C₃₋₁₄-heteroaryl” means an aromatic, monocyclic, bicyclic or tricyclic moiety comprising 3 to 14 ring atoms, wherein each cycle comprises independently of one another 1, 2, 3, 4 or more heteroatoms independently of one another selected from the group consisting of nitrogen, oxygen and sulfur, whereas the remaining ring atoms are carbon atoms, and whereas bicyclic or tricyclic systems may share common heteroatom(s). Examples of preferred -C₃₋₁₄-heteroaryl moieties according to the invention include but are not limited to pyrrole, pyrazole, imidazole, triazole, tetrazole, furane, thiophene, oxazole, isoxazole, thiazole, isothiazole, pyridine, pyridazine, pyrimidine, pyrazine, indolicine, 9H-chinolicine, 1,8-naphthyridine, purine, imidazo[1,2-a]pyrazine, and pteridine. The -C₃₋₁₄-heteroaryl, which is bonded to the compound according to the invention, in its periphery may optionally be condensed with a -C₃₋₁₂-cycloalkyl, saturated or unsaturated, unsubstituted, mono- or polysubstituted; and/or with a -C₃₋₁₂-heterocycloalkyl, saturated or unsaturated, unsubstituted, mono- or polysubstituted; and/or with a -C₆₋₁₄-aryl, unsubstituted, mono- or polysubstituted. Under these circumstances, the ring atoms of the condensed moieties are not included in the 6 to 14 ring carbon atoms of the 6-14-membered heterocycloalkyl moieties. Examples of -C₃₋₁₄-heteroaryl moieties condensed with -C₃₋₁₂-cycloalkyl moieties include but are not limited to 5,6,7,8-tetrahydroquinoline and 5,6,7,8-tetrahydroquinazoline, which in each case are connected through the -C₃₋₁₄-heteroaryl. An examples of a -C₃₋₁₄-heteroaryl condensed with a -C₃₋₁₂-heterocycloalkyl includes but is not limited to 5,6,7,8-tetrahydro[1,2,4]triazolo[1,5-a]pyrazine, which is connected through the -C₃₋₁₄-heteroaryl. Examples of -C₃₋₁₄-heteroaryl moieties condensed with -C₆₋₁₄-aryl moieties include but are not limited to quinoline, isoquinoline, phenazine and phenoxacine, which in each case are connected through the -C₃₋₁₄-heteroaryl.

According to the invention, the -C₃₋₁₄-heteroaryl may optionally be connected through -C₁₋₆-alkylene-, i.e. the -C₃₋₁₄-heteroaryl may not be directly bound to the compound according to general formula (I) but may be connected thereto through a -C₁₋₆-alkylene- linker. Said linker may be connected to a carbon ring atom or to a hetero ring atom of the -C₃₋₁₄-heteroaryl. Examples include but are not limited to —CH₂-oxazole, —CH₂-isoxazole, —CH₂-imidazole, —CH₂-pyridine, —CH₂-pyrimidine, —CH₂-pyridazine, —CH₂CH₂-oxazole, —CH₂CH₂-isoxazole, —CH₂CH₂-imidazole, —CH₂CH₂-pyridine, —CH₂CH₂-pyrimidine, and —CH₂CH₂-pyridazine.

According to the invention, unless expressly stated otherwise, the -C₃₋₁₄-heteroaryl can be unsubstituted, mono- or polysubstituted. Examples of -C₃₋₁₄-heteroaryl moieties include but are not limited to 1-methyl-5-imidazolyl, 3-methyl-5-imidazolyl, 3,5-dimethyl-4-isoxazolyl, 2-amine-3-pyrazinyl, 2-methoxy-4-pyridinyl, 2-methoxy-5-pyridinyl, 3-methoxy-4-pyridinyl, 3-methoxy-6-pyridinyl, 4-methoxy-2-pyridinyl, 2-methylsulfonyl-5-pyridinyl, 3-methylsulfonyl-6-pyridinyl, 3-methoxy-6-pyridazinyl, 2-nitrilo-5-pyrimidinyl, 4-hydroxy-2-pyrimidinyl, 4-methoxy-pyrimidinyl, and 2-methoxy-6-pyrazinyl.

In preferred embodiments, X means —SR0:

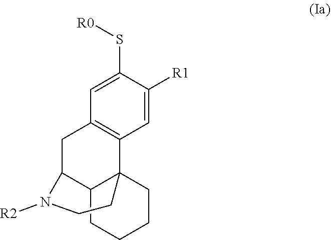

In other preferred embodiments, X means —NR3R4:

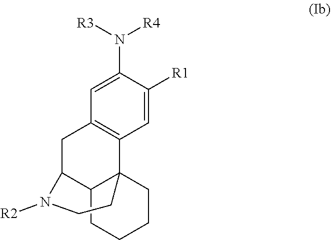

In preferred embodiments, R1 means —OC₁₋₆-alkyl, unsubstituted or substituted, and R2 means —C₁₋₆-alkyl; preferably R1 means —OCH₃ and R2 means —CH₃.

In preferred embodiments, X means —SR0; preferably —S-C₁₋₆-alkyl, unsubstituted or substituted; more preferably, —S—CH₃.

In preferred embodiments, X means —SR0; preferably —S-C₃₋₁₄-heteroaryl, unsubstituted or substituted; more preferably a moiety according to general formula (F) or (G)

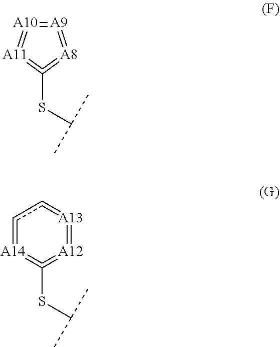

wherein A8, A9, A10, A11, A12, A13 and A14 independently of one another mean —CH═, —C(CH₃)═, —C(NH₂)═, —N═, —NH—, —N(CH₃)—, or —O—.

In particularly preferred embodiments, X means —S-C₃₋₁₄-heteroaryl according to general formula (F), wherein
A8 and A11 mean —C(CH₃)═, A9 means —N═, and A10 means —O—;
A8 means —N(CH₃)—, A9 and A11 mean —CH═, and A10 means —N═;
A8 means —N═, A9 and A11 mean —CH═, and A10 means —N(CH₃)—; or
A8 means —N═, A9 and A11 means —CH═, and A10 means —NH—; or —S-C₃₋₁₄-heteroaryl according to general formula (G), wherein
A12 means —CH═, A13 and A14 mean —N═; or
A12 means —C(NH₂)═, and A13 and A14 mean —N═.

In preferred embodiments, X means —NR3R4 and
R3 means —H, —R0, —C(═O)R0, —C(═O)OR0, —C(═O)NHR0, —C(═O)N(R0)₂, —C(═O)NH₂, —C(═O)CH(NH₂)R0 or —S(═O)₂R0; preferably —H or —R0; more preferably —H or —C₁₋₆-alkyl, unsubstituted or substituted; still more preferably —H or —CH₃; and/or
R4 means —C(═O)R0, —C(═O)OR0, —C(═O)NHR0, —C(═O)N(R0)₂, —C(═O)NH₂, —C(═O)CH(NH₂)R0 or —S(═O)₂R0; preferably —C(═O)R0 or —C(═O)NHR0; more preferably —C(═O)R0 or —C(═O)NH—C₁₋₆-alkyl, unsubstituted or substituted; still more preferably —C(═O)R0 or —C(═O)NH—CH₃.

Preferably, X means —NR3R4 and R3 means —H, —R0, or —C(═O)NHR0; preferably —H, -C₁₋₆-alkyl, unsubstituted or substituted, or —C(═O)NH—C₁₋₆-alkyl, unsubstituted or substituted; more preferably —H, —CH₃, or —C(═O)NH—CH₃.

In preferred embodiments, R3 means —H.

In other preferred embodiments, R3 means —CH₃.

In still other preferred embodiments, R3 means —C(═O)NH—CH₃.

In preferred embodiments,
(i) R1 means —OC₁₋₆-alkyl, unsubstituted or substituted; preferably —OCH₃;
(ii) R2 means —C₁₋₆-alkyl; preferably —CH₃; and
(iii) X means —NR3R4, wherein R3 means —H, -C₁₋₆-alkyl, unsubstituted or substituted, or —C(═O)NHC₁₋₆-alkyl, unsubstituted or substituted; preferably —H, —CH₃, or —C(═O)NH—CH₃.

Preferably, X means —NR3R4;
R3 means —H, —R0, —C(═O)R0, —C(═O)OR0 or —C(═O)NHR0; preferably —H or —CH₃; and
R4 means —C(═O)R0, —C(═O)N(R0)₂, —C(═O)NH₂, —C(═O)CH(NH₂)R0 or —S(═O)₂R0; preferably —C(═O)-C₁₋₆-alkyl, —C(═O)-C₃₋₁₂-cycloalkyl, —C(═O)-C₃₋₁₂-heterocycloalkyl, —C(═O)-C₃₋₁₄-heteroaryl, —C(═O)N(C₁₋₆-alkyl)₂, —C(═O)NH₂, —C(═O)C(NH₂)-C₃₋₁₂-cycloalkyl or —S(═O)₂—C₁₋₆-alkyl, in each case independently unsubstituted or substituted.

In preferred embodiments, R4 means —C(═O)-C₃₋₁₂-cycloalkyl, unsubstituted or substituted, or —C(═O)-C₃₋₁₂-heterocycloalkyl, unsubstituted or substituted; more preferably a moiety according to general formula (A), (B) or (C)

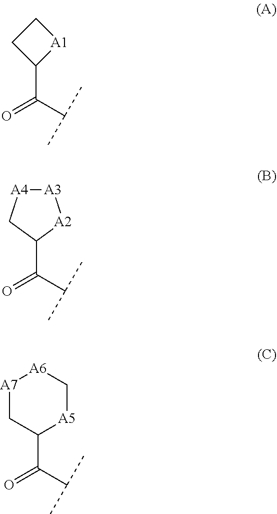

wherein A1, A2, A3, A4, A5, A6 and A7 independently of one another mean —CH₂—, —CHF—, —NH—, —N(CH₃)—, or —O—.

In preferred embodiments, R4 means —C(═O)-C₃₋₁₄-heteroaryl, unsubstituted or substituted; more preferably a moiety according to general formula (D) or (E)

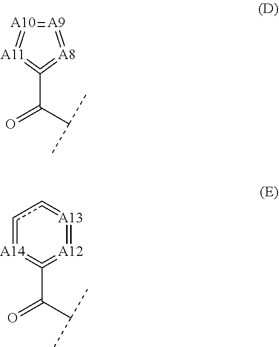

wherein A8, A9, A10, A11, A12, A13 and A14 independently of one another mean —CH═, —C(CH₃)═, —C(NH₂)═, —N═, —NH—, —N(CH₃)—, or —O—.

In particularly preferred embodiments, R4 means
—S(═O)₂—CH₃;
—C(═O)CH₃;
—C(═O)C(CH₃)₃;
—C(═O)—CH₂—CF₃;
—C(═O)N(CH₃)₂;
—C(═O)NH₂;
—C(═O)CH(NH₂)—(C₃H₅-cycloalkyl);
—C(═O)-C₃₋₁₂-cycloalkyl according to general formula (A), wherein
   A1 means —CH₂—; or
—C(═O)-C₃₋₁₂-cycloalkyl according to general formula (B), wherein
   A2, A3, and A4 mean —CH₂—; or
—C(═O)-C₃₋₁₂-heterocycloalkyl according to general formula (A), wherein
   A1 means —NH—; or
—C(═O)-C₃₋₁₂-heterocycloalkyl according to general formula (B), wherein
   A2 means —NH—, and A3 and A4 mean —CH₂—;
   A2 means —N(CH₃)—, and A3 and A4 mean —CH₂—;
   A2 means —O—, and A3 and A4 mean —CH₂—;
   A2 means —NH—, A3 means —CH₂—, and R4 means —CHF—; or
   A2 and A4 mean —CH₂—, and A3 means —NH—; or
—C(═O)-C₃₋₁₂-heterocycloalkyl according to general formula (C), wherein
   A5 means —O—, A6 means —CH₂—, and A7 means —NH—;
   A5 and A7 mean —CH₂—, and A6 means —NH—;
   A5 and A7 mean —CH₂—, and A6 means —O—; or
   A5 means —NH—, A6 means —CH₂—, and A7 means —N(CH₃)—; or
—C(═O)-C₃₋₁₄-heteroaryl according to general formula (D), wherein
   A8 and A11 mean —C(CH₃)═, A9 means —N═, and A10 means —O—;
   A8 means —N(CH₃)—, A9 and A11 mean —CH═, and A10 means —N═;
   A8 means —N═, A9 and A11 mean —CH═, and A10 means —N(CH₃)—; or
   A8 means —N═, A9 and A11 means —CH═, and A10 means —NH—; or
—C(═O)-C₃₋₁₄-heteroaryl according to general formula (E), wherein
   A12 means —CH═, A13 and A14 mean —N═; or
   A12 means —C(NH₂)═, and A13 and A14 mean —N═.

In especially preferred embodiments, X means —NR3R4 with R3 being —H, i.e. —NHR4, wherein R4 means
—C(═O)-C₃₋₁₂-heterocycloalkyl according to general formula (B), wherein A2 means —NH—, and A3 and A4 mean —CH₂—;
—C(═O)-C₃₋₁₂-cycloalkyl according to general formula (B), wherein A2, A3, and A4 mean —CH₂—;
—C(═O)-C₃₋₁₄-heteroaryl according to general formula (D), wherein A8 means —N═, A9 and A11 means —CH═, and A10 means —NH—;
—C(═O)-C₃₋₁₂-heterocycloalkyl according to general formula (A), wherein A1 means —NH—;
—C(═O)-C₃₋₁₂-heterocycloalkyl according to general formula (B), wherein A2 and A4 mean —CH₂—, and A3 means —NH—;
—C(═O)-C₃₋₁₂-heterocycloalkyl according to general formula (B), wherein A2 means —O—, and A3 and A4 mean —CH₂—;
—C(═O)-C₃₋₁₄-heteroaryl according to general formula (D), wherein A8 means —N(CH₃)—, A9 and A11 mean —CH═, and A10 means —N═;
—C(═O)-C₃₋₁₄-heteroaryl according to general formula (D), wherein A8 means —N═, A9 and A11 mean —CH═, and A10 means —N(CH₃)—;
—C(═O)-C₃₋₁₂-heterocycloalkyl according to general formula (C), wherein A5 and A7 mean —CH₂—, and A6 means —O—;
—C(═O)-C₃₋₁₂-heterocycloalkyl according to general formula (B), wherein A2 means —NH—, A3 means —CH₂—, and R4 means —CHF—;
—C(═O)-C₃₋₁₂-heterocycloalkyl according to general formula (B), wherein A2 means —N(CH₃)—, and A3 and A4 mean —CH₂—;
—C(═O)-C₃₋₁₄-heteroaryl according to general formula (D), wherein A8 and A11 mean —C(CH₃)═, A9 means —N═, and A10 means —O—;
—C(═O)-C₃₋₁₂-cycloalkyl according to general formula (A), wherein A1 means —CH₂—;
—C(═O)-C₃₋₁₂-heterocycloalkyl according to general formula (C), wherein A5 means —O—, A6 means —CH₂—, and A7 means —NH—;
—C(═O)-C₃₋₁₂-heterocycloalkyl according to general formula (C), wherein A5 and A7 mean —CH₂—, and A6 means —NH—;
—C(═O)—CH₂—CF₃;
—C(═O)C(CH₃)₃;
—C(═O)CH(NH₂)-cyclopropyl;
—C(═O)-C₃₋₁₄-heteroaryl according to general formula (E), wherein A12 means —CH═, A13 and A14 mean —N═;
—C(═O)-C₃₋₁₄-heteroaryl according to general formula (E), wherein A12 means —C(NH₂)═, and A13 and A14 mean —N═;
—C(═O)-C₃₋₁₂-heterocycloalkyl according to general formula (C), wherein A5 means —NH—, A6 means —CH₂—, and A7 means —N(CH₃)—; or
—S(═O)₂—CH₃.

In especially preferred embodiments, X means —NR3R4 with R3 being —CH₃, i.e. —N(CH₃)R4, wherein R4 means
—C(═O)CH₃;
—C(═O)NH₂;
—C(═O)NH(CH₃);
—C(═O)N(CH₃)₂; or
—S(═O)₂—CH₃.

In preferred embodiments, R3 and R4 together with the nitrogen atom to which they are attached form
an azetidine ring, which is monosubstituted with —N(CH₃)₂ or —SO₂CH₃, or disubstituted with —F;
a pyrrolidine ring, which is monosubstituted with —N(CH₃)₂ or —NH₂;
a piperidine ring, which is unsubstituted or monosubstituted with —C(═O)NHCH₃ or —NH₂;
a piperazine ring, which is disubstituted with ═O and —CH₃;
a pyrazole ring, which is unsubstituted;
a triazole ring, preferably a 1,2,4-triazole ring, which is unsubstituted;
an imidazole ring, which is monosubstituted with —C(═O)NH₂; or
a spirocyclic compound/moiety consisting of an azetidine ring and an oxetanyl ring, which is unsubstituted.

In preferred embodiments, the compound according to the invention has a stereochemistry according to general formula (II)

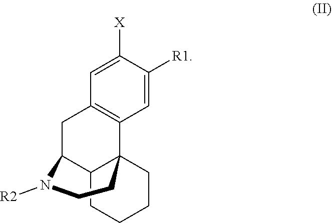

If the compounds according to the invention are chiral, then they are preferably present as racemate or a mixture of stereoisomers or diastereomers or in enriched form of an enantiomer. In a preferred embodiment the enantiomer excess (ee) of the S-enantiomer amounts to at least 50% ee, more preferred at least 75% ee, more preferred at least 90% ee, most preferred at least 95% ee, and in particular at least 99% ee. In another preferred embodiment, the enantiomer excess (ee) of the R-enantiomer amounts to at least 50% ee, more preferred at least 75% ee, more preferred at least 90% ee, most preferred at least 95% ee, and in particular at least 99% ee.

The morphinan-derivatives have chiral centers and can therefore occur in various stereoisomeric forms. The general formula (I) encompasses all these forms.

Suitable methods for separating the enantiomers are known to the person skilled in the art. Preparative HPLC on chiral stationary phases and conversion into diastereomeric intermediates can be given as examples. The conversion into diastereomeric intermediates can occur, for example, as salt formation by means of chiral, enantiomer-pure acids. After separation of the diastereomers thus formed, the salt can then be converted into the free base or another salt again.

Unless expressly specified, each reference to the compounds according to the invention covers all isomers in pure form and admixture with one another (e.g. stereoisomers, diastereomers, enantiomers) in any desired mixture ratio.

In preferred embodiments, the compound according to the invention is selected from the group consisting of

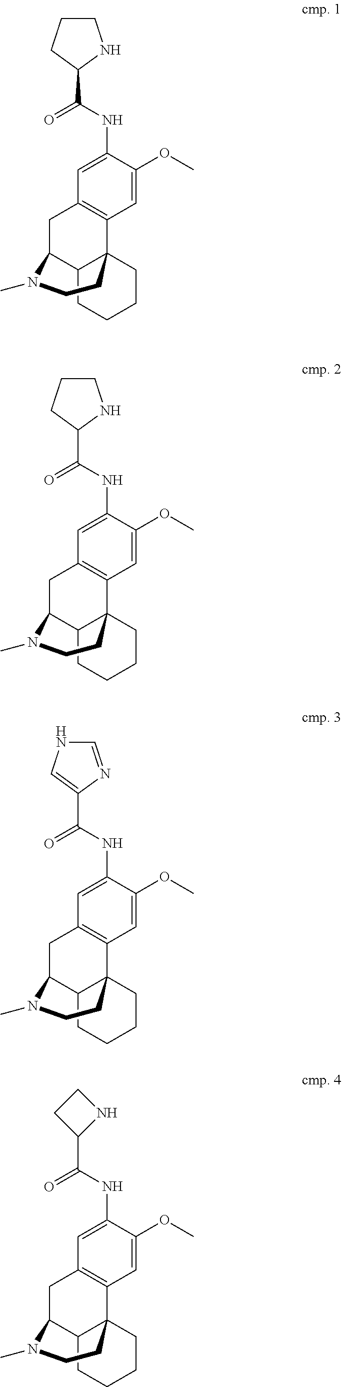

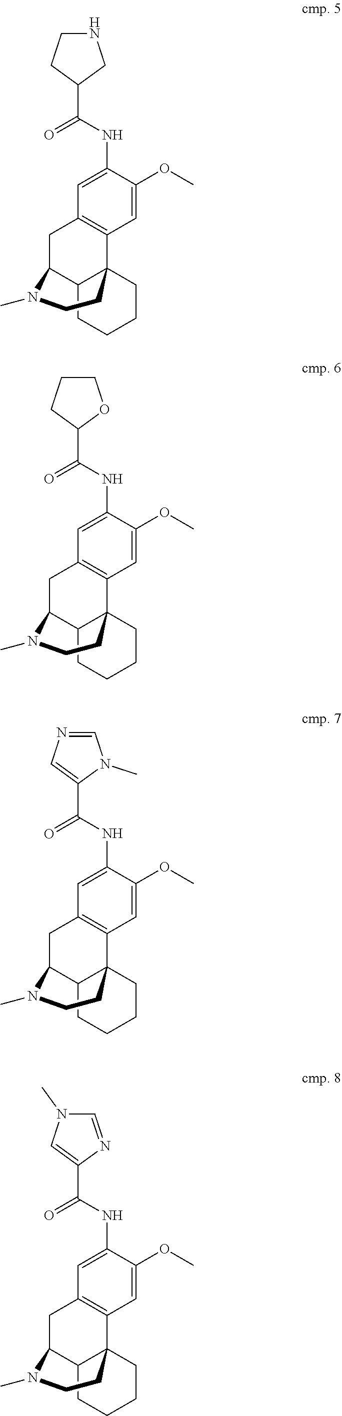

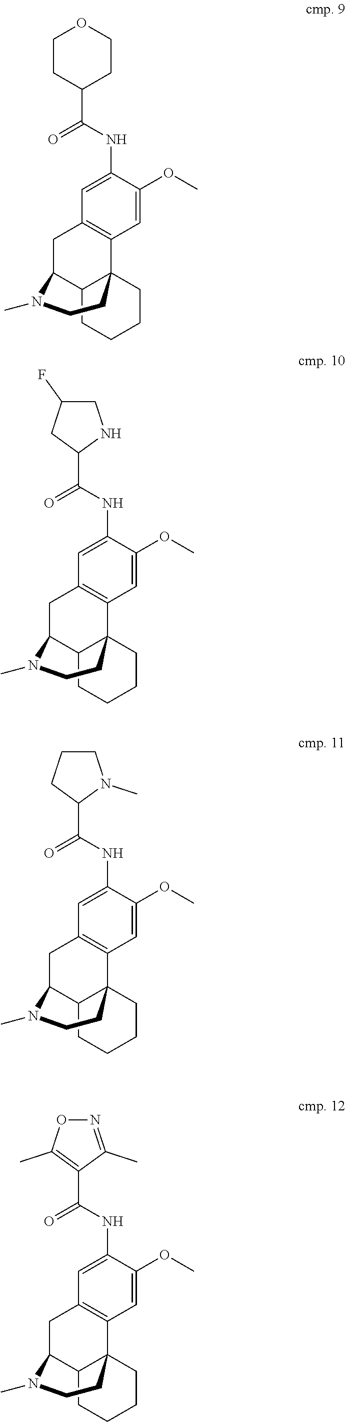

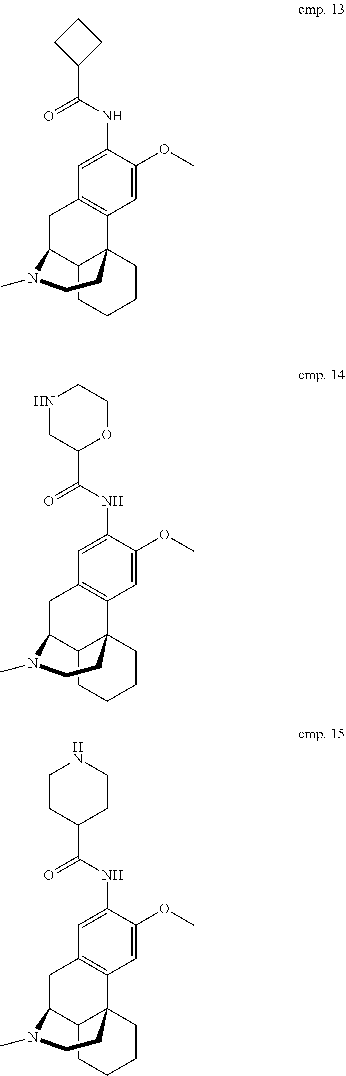

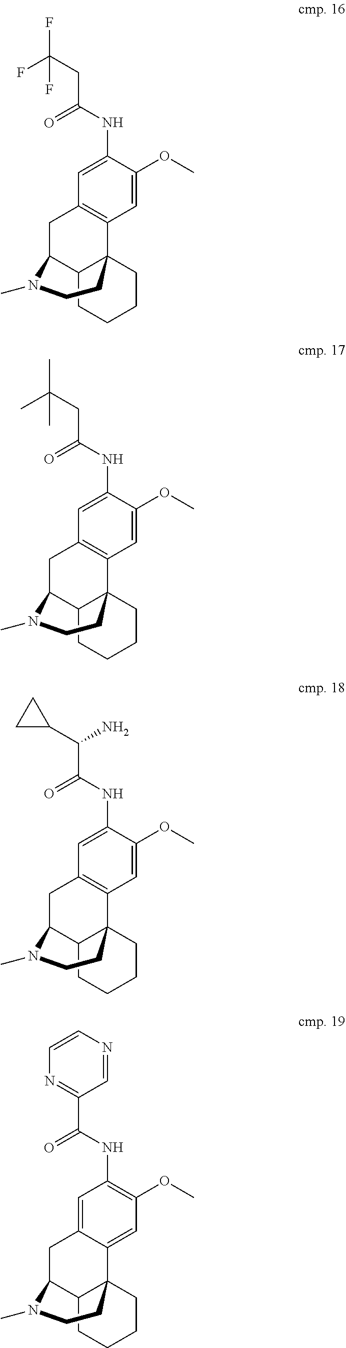

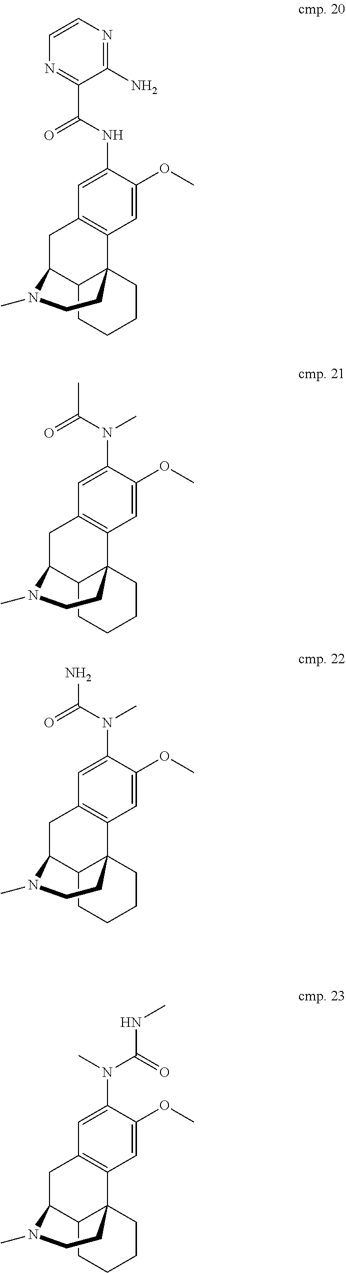

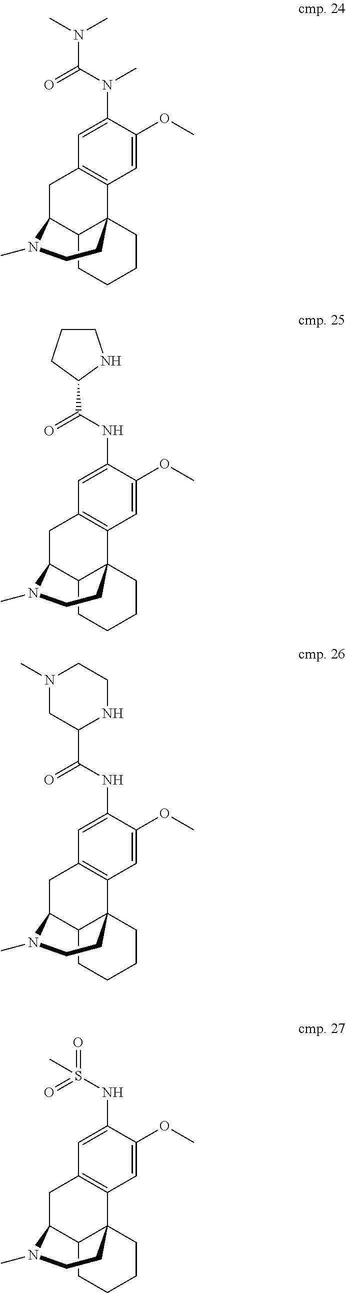

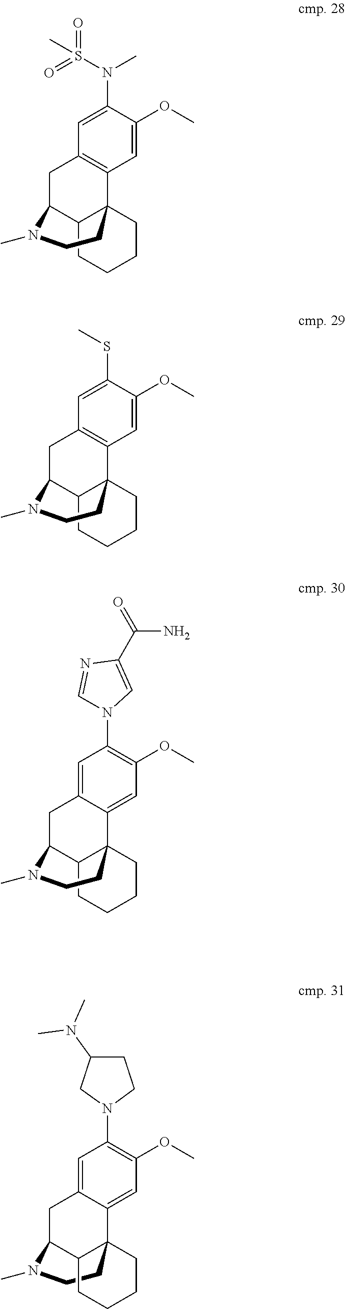

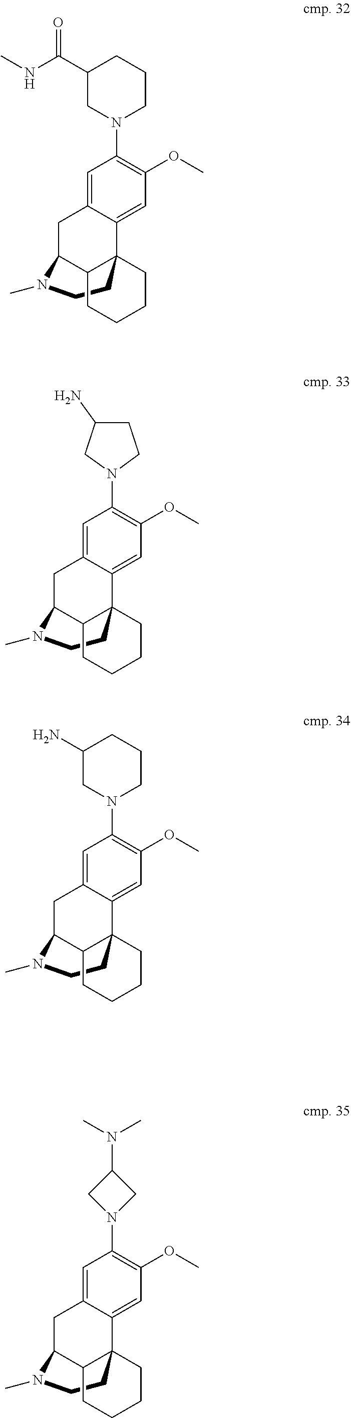

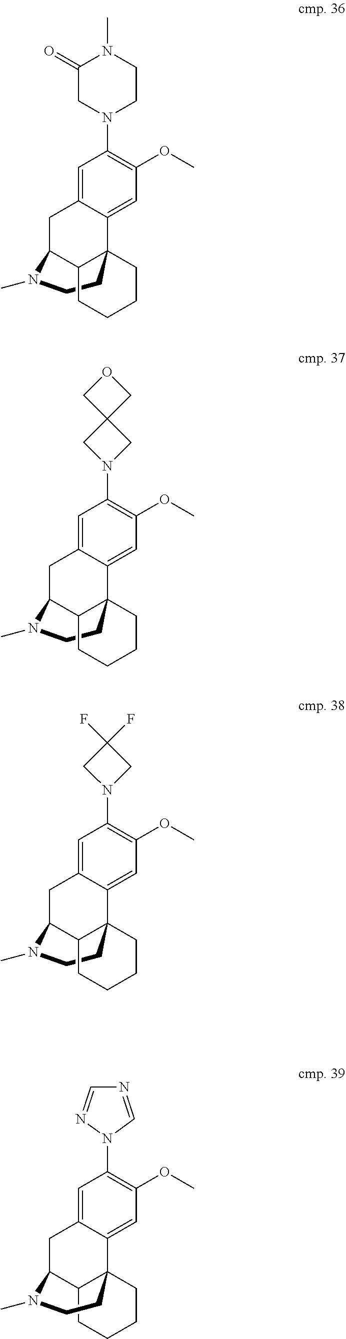

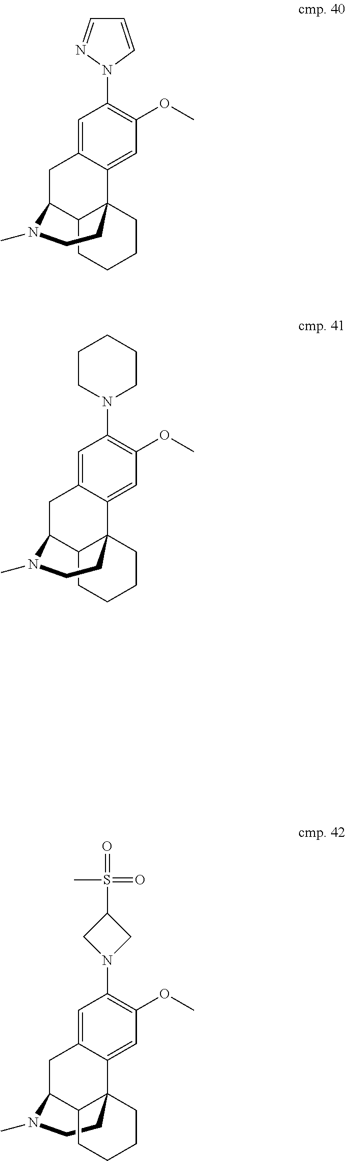

or a physiologically acceptable salt thereof.

The above compounds usually or preferably rotate polarized light in (+)-direction (dextrorotatory).

In a preferred embodiment, X does not mean —SCF₃, —SC₆H₅, or —N(H)(C(═O)CH₃), i.e. —N(H)(Ac); and R2 does not mean —H or —C(═O)—O—CH₂—C₆H₅, i.e. —(Cbz).

In a preferred embodiment, (i) X is defined according to paragraphs [0058] and [0059] above; or (ii) X means —S-C₁₋₆-alkyl, unsubstituted, preferably —S—CH₃; and R2 means —C₁₋₆-alkyl, unsubstituted, preferably —CH₃.

In a preferred embodiment and in the context of paragraph [0068] above, R2 means —C₁₋₆-alkyl, unsubstituted, preferably —CH₃ and/or R1 means —OC₁₋₆-alkyl, unsubstituted, preferably —OCH₃.

Preferably, the compound according to the invention is selected from the group consisting of
(2R)-N-[(1S,9S)-4-methoxy-17-methyl-17-azatetracyclo[7.5.3.0¹,¹⁰,0²,⁷]heptadeca-2(7),3,5-trien-5-yl]pyrrolidine-2-carboxamide; cmp. 1;
N-[(1S,9S)-4-methoxy-17-methyl-17-azatetracyclo[7.5.3.0¹,¹⁰,0²,⁷]heptadeca-2(7),3,5-trien-5-yl]cyclopentanecarboxamide; cmp. 2;
N-[(1S,9S)-4-methoxy-17-methyl-17-azatetracyclo[7.5.3.0¹,¹⁰,0²,⁷]heptadeca-2(7),3,5-trien-5-yl]-1H-imidazole-4-carboxamide; cmp. 3;
N-[(1S,9S)-4-methoxy-17-methyl-17-azatetracyclo[7.5.3.0¹,¹⁰,0²,⁷]heptadeca-2(7),3,5-trien-5-yl]azetidine-2-carboxamide; cmp. 4;
N-[(1S,9S)-4-methoxy-17-methyl-17-azatetracyclo[7.5.3.0¹,¹⁰,0²,⁷]heptadeca-2(7),3,5-trien-5-yl]pyrrolidine-3-carboxamide; cmp. 5;
N-[(1S,9S)-4-methoxy-17-methyl-17-azatetracyclo[7.5.3.0¹,¹⁰,0²,⁷]heptadeca-2(7),3,5-trien-5-yl]oxolane-2-carboxamide; cmp. 6;
N-[(1S,9S)-4-methoxy-17-methyl-17-azatetracyclo[7.5.3.0¹,¹⁰,0²,⁷]heptadeca-2(7),3,5-trien-5-yl]-1-methyl-1H-imidazole-5-carboxamide; cmp. 7;
N-[(1S,9S)-4-methoxy-17-methyl-17-azatetracyclo[7.5.3.0¹,¹⁰,0²,⁷]heptadeca-2(7),3,5-trien-5-yl]-1-methyl-1H-imidazole-4-carboxamide; cmp. 8;
N-[(1S,9S)-4-methoxy-17-methyl-17-azatetracyclo[7.5.3.0¹,¹⁰,0²,⁷]heptadeca-2(7),3,5-trien-5-yl]oxane-4-carboxamide; cmp. 9;
4-fluoro-N-[(1S,9S)-4-methoxy-17-methyl-17-azatetracyclo[7.5.3.0¹,¹⁰,0²,⁷]heptadeca-2(7),3,5-trien-5-yl]pyrrolidine-2-carboxamide; cmp. 10;
N-[(1S,9S)-4-methoxy-17-methyl-17-azatetracyclo[7.5.3.0¹,¹⁰,0²,⁷]heptadeca-2(7),3,5-trien-5-yl]-1-methylpyrrolidine-2-carboxamide; cmp. 11;
N-[(1S,9S)-4-methoxy-17-methyl-17-azatetracyclo[7.5.3.0¹,¹⁰,0²,⁷]heptadeca-2(7),3,5-trien-5-yl]-3,5-dimethyl-1,2-oxazole-4-carboxamide; cmp. 12;
N-[(1S,9S)-4-methoxy-17-methyl-17-azatetracyclo[7.5.3.0¹,¹⁰,0²,⁷]heptadeca-2(7),3,5-trien-5-yl]cyclobutanecarboxamide; cmp. 13;
N-[(1S,9S)-4-methoxy-17-methyl-17-azatetracyclo[7.5.3.0¹,¹⁰,0²,⁷]heptadeca-2(7),3,5-trien-5-yl]morpholine-2-carboxamide; cmp. 14;
N-[(1S,9S)-4-methoxy-17-methyl-17-azatetracyclo[7.5.3.0¹,¹⁰,0²,⁷]heptadeca-2(7),3,5-trien-5-yl]piperidine-4-carboxamide; cmp. 15;
3,3,3-trifluoro-N-[(1S,9S)-4-methoxy-17-methyl-7-azatetracyclo[7.5.3.0¹,¹⁰,0²,⁷]heptadeca2(7),3,5-trien-5-yl]propanamide; cmp. 16;
N-[(1S,9S)-4-methoxy-17-methyl-17-azatetracyclo[7.5.3.0¹,¹⁰,0²,⁷]heptadeca-2(7),3,5-trien-5-yl]-2,2-dimethylpropanamide; cmp. 17;
(2S)-2-amino-2-cyclopropyl-N-[(1S,9S)-4-methoxy-17-methyl-17-azatetracyclo[7.5.3.0¹,¹⁰,0²,⁷]heptadeca-2(7),3,5-trien-5-yl]acetamide; cmp. 18;
N-[(1S,9S)-4-methoxy-17-methyl-17-azatetracyclo[7.5.3.0¹,¹⁰,0²,⁷]heptadeca-2(7),3,5-trien-5-yl]pyrazine-2-carboxamide; cmp. 19;
3-amino-N-[(1S,9S)-4-methoxy-17-methyl-17-azatetracyclo[7.5.3.0¹,¹⁰,0²,⁷]heptadeca-2(7),3,5-trien-5-yl]pyrazine-2-carboxamide; cmp. 20;
N-[(1S,9S)-4-methoxy-17-methyl-17-azatetracyclo[7.5.3.0¹,¹⁰,0²,⁷]heptadeca-2(7),3,5-trien-5-yl]-N-methylacetamide; cmp. 21;
1-[(1S,9S)-4-methoxy-17-methyl-17-azatetracyclo[7.5.3.0¹,¹⁰,0²,⁷]heptadeca-2(7),3,5-trien-5-yl]-1-methylurea; cmp. 22;
1-[(1S,9S)-4-methoxy-17-methyl-17-azatetracyclo[7.5.3.0¹,¹⁰,0²,⁷]heptadeca-2(7),3,5-trien-5-yl]-1,3-dimethylurea; cmp. 23;
1-[(1S,9S)-4-methoxy-17-methyl-17-azatetracyclo[7.5.3.0¹,¹⁰,0²,⁷]heptadeca-2(7),3,5-trien-5-yl]-1,3,3-trimethylurea; cmp. 24;
(2S)—N-[(1S,9S)-4-methoxy-17-methyl-17-azatetracyclo[7.5.3.0¹,¹⁰,0²,⁷]heptadeca-2(7),3,5-trien-5-yl]pyrrolidine-2-carboxamide; cmp. 25;
N-[(1S,9S)-4-methoxy-17-methyl-17-azatetracyclo[7.5.3.0¹,¹⁰,0²,⁷]heptadeca-2(7),3,5-trien-5-yl]-4-methylpiperazine-2-carboxamide; cmp. 26;
N-[(1S,9S)-4-methoxy-17-methyl-17-azatetracyclo[7.5.3.0¹,¹⁰,0²,⁷]heptadeca-2(7),3,5-trien-5-yl]methanesulfonamide; cmp. 27;
N-[(1S,9S)-4-methoxy-17-methyl-17-azatetracyclo[7.5.3.0¹,¹⁰,0²,⁷]heptadeca-2(7),3,5-trien-5-yl]methanesulfonamide; cmp. 28;
(1S,9S)-4-methoxy-17-methyl-5-(methylsulfanyl)-17-azatetracyclo[7.5.3.0¹,¹⁰,0²,⁷]heptadeca2(7),3,5-triene; cmp. 29;
1-[(1S,9S)-4-methoxy-17-methyl-17-azatetracyclo[7.5.3.0¹,¹⁰,0²,⁷]heptadeca-2,4,6-trien-5-yl]-1Himidazole-4-carboxamide; cmp. 30;
1-[(1S,9S)-4-methoxy-17-methyl-17-azatetracyclo[7.5.3.0¹,¹⁰,0²,⁷]heptadeca-2(7),3,5-trien-5-yl]-N,N-dimethylpyrrolidin-3-amine; cmp. 31;
1-[(1S,9S)-4-methoxy-17-methyl-17-azatetracyclo[7.5.3.0¹,¹⁰,0²,⁷]heptadeca-2(7),3,5-trien-5-yl]-N-methylpiperidine-3-carboxamide; cmp. 32;
1-[(1S,9S)-4-methoxy-17-methyl-17-azatetracyclo[7.5.3.0¹,¹⁰,0²,⁷]heptadeca-2(7),3,5-trien-5-yl]pyrrolidin-3-amine; cmp. 33;
1-[(1S,9S)-4-methoxy-17-methyl-17-azatetracyclo[7.5.3.0¹,¹⁰,0²,⁷]heptadeca-2(7),3,5-trien-5-yl]piperidin-3-amine; cmp. 34;
1-[(1S,9S)-4-methoxy-17-methyl-17-azatetracyclo[7.5.3.0¹,¹⁰,0²,⁷]heptadeca-2(7),3,5-trien-5-yl]-N,N-dimethylazetidin-3-amine; cmp. 35;
4-[(1S,9S)-4-methoxy-17-methyl-17-azatetracyclo[7.5.3.0¹,¹⁰,0²,⁷]heptadeca-2(7),3,5-trien-5-yl]-1-methylpiperazin-2-one; cmp. 36;
(1S,9S)-4-methoxy-17-methyl-5-{2-oxa-6-azaspiro[3.3]heptan-6-yl}-17-azatetracyclo[7.5.3.0¹,¹⁰,0²,⁷]heptadeca-2(7),3,5-triene; cmp. 37;
(1S,9S)-5-(3,3-difluoroazetidin-1-yl)-4-methoxy-17-methyl-17-azatetracyclo[7.5.3.0¹,¹⁰,0²,⁷]heptadeca-2(7),3,5-triene; cmp. 38;
(1S,9S)-4-methoxy-17-methyl-5-(1H-1,2,4-triazol-1-yl)-17-azatetracyclo[7.5.3.0¹,¹⁰,0²,⁷]heptadeca2,4,6-triene; cmp. 39;
(1S,9S)-4-methoxy-17-methyl-5-(1H-pyrazol-1-yl)-17-azatetracyclo[7.5.3.0¹,¹⁰,0²,⁷]heptadeca2,4,6-triene; cmp. 40;
(1S,9S)-4-methoxy-17-methyl-5-(piperidin-1-yl)-17-azatetracyclo[7.5.3.0¹,¹⁰,0²,⁷]heptadeca-2,4,6-triene; cmp. 41;
(1S,9S)-5-(3-methanesulfonylazetidin-1-yl)-4-methoxy-17-methyl-17-azatetracyclo[7.5.3.0¹,¹⁰,0²,⁷]heptadeca-2(7),3,5-triene; cmp. 42;
and/or a physiologically acceptable salt thereof.

The compound N-[(1S,9S)-4-methoxy-17-methyl-17-azatetracyclo[7.5.3.01,10.02,7]-heptadeca-2,4,6-trien-5-yl]-1-methyl-1H-imidazole-5-carboxamide is particularly preferred.

Particularly preferably, the morphinan-derivative is a (+)-morphinan-derivative.

In preferred embodiments, the compounds according to the invention are in the form of the free bases.

In other preferred embodiments, the compounds according to the invention are in the form of physiologically acceptable salts.

Unless expressly specified, each reference to the compounds according to the invention covers the free compounds (i.e. the forms that are not present in the form of salt) and all physiologically compatible salts.

As used herein, compounds are morphinans.

Preferably, the compounds according to the invention are blood brain barrier-impermeable derivatives of dextromethorphan.

Preferably, the compounds according to the invention do not bind to hERG (potassium voltage-gated channel subfamily H member 2) and/or -type opioid (MOP) receptor.

The morphinan-derivatives according to the invention and also the starting materials for their preparation are prepared by methods known per se, as described in the literature (for example in the standard works, such as Houben-Weyl, Methoden der organischen Chemie [Methods of Organic Chemistry], Georg-Thieme-Verlag, Stuttgart), under reaction conditions, which are known and suitable for the said reactions. Use can also be made here of variants known per se, which are not mentioned here in greater detail.

If desired, the starting materials can also be formed in situ so that they are not isolated from the reaction mixture, but instead are immediately converted further into the morphinan-derivatives according to the invention.

The starting compounds are generally known. If they are novel, however, they can be prepared by methods known per se.

In preferred embodiments, the synthesis of the compounds according to the invention proceeds via a synthesis route which comprises the preparation of an intermediate according to general formula (III)

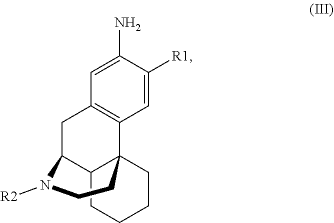

wherein R1 and R2 are defined as above;
or a physiologically acceptable salt thereof.

The morphinan-derivatives according to the invention can be used in their final non-salt form. On the other hand, the invention also encompasses the use of these morphinan-derivatives in the form of their pharmaceutically acceptable salts, which can be derived from various organic and inorganic acids and bases by procedures known in the art. Pharmaceutically acceptable salt forms of the morphinan-derivatives are for the most part prepared by conventional methods. If the morphinan-derivative contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the morphinan-derivatives are likewise included.

In the case of certain morphinan-derivatives, acid-addition salts can be formed by treating the morphinan-derivatives with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the morphinan-derivatives include, but are not limited to acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, and phthalate.

Furthermore, the base salts of the morphinan-derivatives according to the invention include, but are not limited to aluminium, ammonium, calcium, copper, iron(III), iron(II), lithium, magnesium, manganese(III), manganese(II), potassium, sodium and zinc salts, but this is not intended to represent a restriction. Salts of the morphinan-derivatives which are derived from pharmaceutically acceptable organic non-toxic bases include, but are not limited to salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methylD-glucamine, morpholine, piperazine, piperidine, polyamine resins, procaine, purines, theobromine, triethanolamine, triethylamine, trimethylamine, tripropylamine and tris(hydroxymethyl)methylamine (tromethamine).

The morphinan-derivatives of the invention may contain basic nitrogen-containing groups that can be quaternized using agents such as -C₁-C₄-alkyl halides, for example -methyl, -ethyl, -isopropyl and -tert-butyl, -chloride, -bromide and -iodide; -di(C₁-C₄)alkyl sulfates, for example -dimethyl, -diethyl and -diamyl sulfate; -(C₁₀-C₁₈)alkyl halides, for example -decyl, -dodecyl, -lauryl, -myristyl - and stearyl chloride, bromide and iodide; and -aryl(C₁-C₄)alkyl halides, for example -benzyl chloride and -phenethyl bromide. Both water- and oil-soluble morphinan-derivatives according to the invention can be prepared using such salts.

The above-mentioned pharmaceutical salts which are preferred include, but are not limited to acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine. The hydrochloride, hydrobromide and citrate are preferred.

The acid-addition salts of basic morphinan-derivatives are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

The pharmaceutically acceptable base-addition salts ofthe morphinan-derivatives are preferably formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic morphinan-derivatives according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a morphinan-derivative according to the invention contains more than one group, which is capable of forming pharmaceutically acceptable salts of this type, the invention also encompasses multiple salts. Typical multiple salt forms include, but are not limited to bitartrate, diacetate, difumarate, dimeglumine, diphosphate, disodium, dihydrobromide, trihydrobromide, dihydrochloride, and trihydrochloride.

Accordingly, the expression “pharmaceutically/physiologically acceptable salt” for the purpose of the specification means an active ingredient which comprises a morphinan-derivative in the form of one of its salts, in particular if this salt form imparts improved pharmacokinetic properties on the active ingredient compared with the free form of the active ingredient or any other salt form of the active ingredient used earlier. The pharmaceutically/physiologically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

The morphinan-derivatives according to the invention are chiral owing to their molecular structure and may accordingly occur in various enantiomeric forms. They therefore exist in racemic or in optically active form. Since the pharmaceutical activity of the racemates or stereoisomers of the morphinan-derivatives according to the invention may differ, it may be desirable to use the enantiomers.

In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilized on silica gel). Suitable eluents for this purpose are aqueous or alcoholic solvent mixtures, such as, for example, hexane/isopropanol/acetonitrile, for example in the ratio 82:15:3.

Another aspect of the invention relates to the morphinan-derivatives according to the invention as medicaments and/or medicament active ingredients, preferably for use in the treatment and/or prophylaxis of diseases or conditions selected from insulin-dependent diabetes mellitus, non-insulin-dependent diabetes mellitus; obesity; neuropathy and/or nephropathy, preferably diabetic nephropathy; cancer (in particular neuroendocrine tumors, pancreatic ductal carcinoma, breast and prostate cancer, ovarian cancer and glioma in most of which expression of NMDA receptors was shown to be expressed or correlated with a bad prognosis) (Li & Hanahan in Cell 2013, 153: 86-100), coronary heart disease and stroke, diabetic long-term complications (such as diabetic nephropathy, diabetic neuropathy, diabetic retinopathy, stroke, myocardial infarction, etc.) as well as ischemia-reperfusion injuries (that occur in percutaneous coronary intervention, application of stents to open arteries, and organ transplantation).

Another aspect of the invention relates to the use of the morphinan-derivatives according to the invention for the preparation of a pharmaceutical composition or pharmaceutical dosage form for the treatment and/or prophylaxis of the said diseases or conditions.

Another aspect of the invention relates to a method for the treatment and/or prophylaxis of said diseases or conditions which comprises the administration of an effective amount of one or more morphinan-derivatives according to the invention to a subject in need of such an administration.

The morphinan-derivatives according to the invention are not only useful for the treatment of insulin-dependent diabetes mellitus and its complications, but also for the treatment of non-insulin-dependent diabetes mellitus. This is because patients with non-insulin-dependent diabetes mellitus do also profit from beta-cell-stimulating therapies. As a matter of fact, the World Health Organization (WHO) placed the oral antidiabetic drug glibenclamide in their 17th edition of Essential Medicine in category 18.5, Insulin and other medicines usedfor diabetes. Glibenclamide stimulates insulin secretion from pancreatic islets of non-insulin-dependent diabetic patients, in particular type II diabetics. However, since glibenclamide stimulates basal insulin secretion from pancreatic islets to a large extent, hypoglycemic adverse events are encountered by this drug. In contrast to glibenclamide, however, the morphinan-derivatives according to the invention such as morphinan stimulate basal insulin secretion from pancreatic islets to a lesser extent. Thus, the morphinan-derivatives according to the invention likely have lesser hypoglycemic adverse effects compared to glibenclamide.

The morphinan-derivatives according to the invention are useful for treating diabetes mellitus type 2 and its complications; particularly in overweight patients, when dietary management and exercise alone does not result in adequate glycemic control. The morphinan-derivatives may be used as monotherapy or in combination with other oral antidiabetic agents such as metformin, DPP-4 inhibitors (e.g. sitagliptin, vildagliptin), SGLT-2 inhibitors (e. g. empagliflozin, dapagliflozin), insulin sensitizers (e.g. pioglitazone, rosiglitazone), or with insulin and incretin-like drugs (e.g. exendin-4, liraglutide). The morphinan-derivatives might be orally applied or injected.

The host or patient may belong to any mammal species, for example a primate species, particularly humans; rodents, including mice, rats and hamsters; rabbits; horses, pigs, cows, dogs, cats, etc.

Animal models are of interest for experimental investigations, where they provide a model for the treatment of a human disease.

The morphinan-derivatives according to the invention also mean the physiologically acceptable derivatives and solvates.

The invention also relates to the stereoisomers and the hydrates and solvates of these morphinan-derivatives. Solvates of the morphinan-derivatives include adductions of inert solvent molecules onto the morphinan-derivatives which form owing to their mutual attractive force. Solvates are, for example, mono- or dihydrates or alcoholates.

The morphinan-derivatives include the physiologically acceptable salts (such as HCl and HBr salts) of the morphinan-derivatives according to the invention and also the prodrugs thereof.

Prodrugs mean morphinan-derivatives which have been modified, with, for example, alkyl or acyl groups, sugars or oligopeptides and which are rapidly cleaved in the organism to form the active morphinan-derivatives according to the invention. These also include biodegradable polymer derivatives of the morphinan-derivatives according to the invention.

The invention also includes isotopic isomers of a compound according to the invention, wherein at least one atom of the compound is replaced by an isotope of the respective atom which is different from the naturally predominantly occurring isotope, as well as any mixtures of isotopic isomers of such a compound. Preferred isotopes are ²H (deuterium), ³H (tritium), ¹³C and ¹⁴C.

The expression “effective amount” means the amount of a medicament or pharmaceutical active ingredient which causes a biological or medical response which is sought or aimed at, for example by a researcher or physician, in a tissue, system, animal or human.

In addition, the expression “therapeutically effective amount” means an amount which, compared with a corresponding subject who has not received this amount, has the following consequence: improved treatment, healing, prevention or elimination of a disease, syndrome, condition, complaint, disorder or prevention of side effects or also the reduction in the progress of a disease, condition, disorder or side effects or also the reduction in the progress of a disease, condition or disorder. The expression “therapeutically effective amount” also encompasses the amounts which are effective for increasing normal physiological function.

The invention furthermore relates to the use of the morphinan-derivatives and/or physiologically acceptable salts thereof for the preparation of medicament (pharmaceutical composition), in particular by non-chemical methods. They can be converted into a suitable dosage form here together with at least one solid, liquid and/or semi-liquid excipient or adjuvant and, if desired, in combination with one or more further active ingredients.

The invention furthermore relates to medicaments comprising at least one morphinan-derivative according to the invention and/or physiologically acceptable salts and stereoisomers thereof, including mixtures thereof in all ratios, and optionally excipients and/or adjuvants.

Pharmaceutical compositions can be administered in the form of pharmaceutical dosage forms which comprise a predetermined amount of active ingredient per pharmaceutical dosage forms. Such a unit can comprise, for example, 0.5 mg to 5 g, preferably 2 mg to 2 g, particularly preferably 5 mg to 500 mg, of a morphinan-derivative according to the invention, depending on the disease condition treated, the method of administration and the age, weight and condition of the patient, or pharmaceutical compositions can be administered in the form of pharmaceutical dosage forms which comprise a predetermined amount of morphinan-derivative per pharmaceutical dosage form. Preferred pharmaceutical dosage forms or pharmaceutical compositions are those which comprise a daily dose or part-dose, as indicated above, or a corresponding fraction thereof of an active ingredient. Furthermore, pharmaceutical compositions of this type can be prepared using a process which is generally known in the pharmaceutical art. For comparison, the anti-diabetic drugs metformin and empagliflozin are currently administered in units of 500 mg to 1 g and 10 mg to 25 mg, respectively.

Pharmaceutical compositions can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such pharmaceutical compositions can be prepared using all processes known in the pharmaceutical art by, for example, combining the active ingredient with the excipient(s) or adjuvant(s).

Pharmaceutical compositions adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the morphinan-derivative can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavor, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubilizer, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinyl-pyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The morphinan-derivatives according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different pharmaceutical dosage forms.

Oral liquids, such as, for example, solution, syrups and elixirs, can be prepared in the form of pharmaceutical dosage forms so that a given quantity comprises a pre specified amount of the morphinan-derivatives. Syrups can be prepared by dissolving the morphinan-derivatives in an aqueous solution with a suitable flavour, while elixirs are prepared using a non-toxic alcoholic vehicle. Suspensions can be formulated by dispersion of the morphinan-derivatives in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The pharmaceutical dosage forms pharmaceutical compositions for oral administration can, if desired, be encapsulated in microcapsules. The pharmaceutical composition can also be prepared in such a way that the release is extended or retarded, such as, for example, by coating or embedding of particulate material in polymers, wax and the like.

The morphinan-derivatives according to the invention and salts, solvates and derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The morphinan-derivatives according to the invention and the salts, solvates and derivatives thereof can also be delivered using monoclonal antibodies as individual carriers to which the morphinan-derivatives are coupled. The morphinan-derivatives can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The morphinan-derivatives may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical compositions adapted for transdermal administration can be administered as independent plasters for extended, close contact with the epidermis of the recipient. Thus, for example, the morphinan-derivatives can be delivered from the plaster by iontophoresis.

Pharmaceutical compositions adapted for topical administration can be formulated as ointments, creams, suspensions, lotions, powders, solutions, pastes, gels, sprays, aerosols or oils.

Pharmaceutical compositions adapted for topical application in the mouth encompass lozenges, pastilles and mouthwashes.

Pharmaceutical compositions adapted for rectal administration can be administered in the form of suppositories or enemas.

Pharmaceutical compositions adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable pharmaceutical compositions for administration as nasal spray or nose drops with a liquid as carrier substance encompass solutions of the morphinan-derivatives in water or oil.

Pharmaceutical compositions adapted for administration by inhalation encompass finely particulate dusts or mists, which can be generated by various types of pressurized dispensers with aerosols, nebulizers or insufflators.

Pharmaceutical compositions adapted for vaginal administration can be administered as pessaries, tampons, creams, gels, pastes, foams or spray pharmaceutical compositions.

Pharmaceutical compositions adapted for parenteral administration include aqueous and non-aqueous sterile injection solutions comprising antioxidants, buffers, bacteriostatics and solutes, by means of which the pharmaceutical composition is rendered isotonic with the blood of the recipient to be treated; and aqueous and non-aqueous sterile suspensions, which may comprise suspension media and thickeners. The pharmaceutical compositions can be administered in single-dose or multidose containers, for example sealed ampoules and vials, and stored in freeze-dried (lyophilized) state, so that only the addition of the sterile carrier liquid, for example water for injection purposes, immediately before use is necessary.

Injection solutions and suspensions prepared in accordance with the recipe can be prepared from sterile powders, granules and tablets.

Pharmaceutical compositions for parenteral administration are preferably administered by injection or infusion, preferably intravenously, intramuscularly, subcutaneously, or the like.

In addition to the above particularly mentioned constituents, the pharmaceutical compositions may also comprise other agents usual in the art with respect to the particular type of pharmaceutical composition; thus, for example, pharmaceutical compositions which are suitable for oral administration may comprise flavours.

A therapeutically effective amount of a morphinan-derivative of the invention depends on a number of factors, including, for example, the age and weight of the human or animal, the precise disease condition which requires treatment, and its severity, the nature of the pharmaceutical composition and the method of administration, and is ultimately determined by the treating physician or veterinarian.

However, an effective amount of a morphinan-derivative according to the invention is generally in the range from 0.05 to 100 mg/kg of body weight of the recipient (mammal) per day and particularly typically in the range from 0.15 to 15 mg/kg of body weight per day. Thus, the actual amount per day for an adult mammal weighing 70 kg is usually between 10 mg and 1000 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the morphinan-derivative according to the invention per se. It can be assumed that similar doses are suitable for the treatment of other conditions mentioned above. For comparison, the daily dose of metformin and empagliflozin used in type 2 diabetic patients is 500 mg to 3 g and 10 mg to 25 mg, respectively.

In a particularly preferred embodiment, the morphinan-derivative according to the invention is administered once daily, or twice daily, or thrice daily, or four times daily, the individually administered dose per administration being within the range of 10±5 mg, or 30±15 mg, or 60±15 mg, or 90±15 mg, or 120±15 mg, or 150±15 mg, or 180±15 mg, or 210±15 mg, or 240±15 mg, or 270±15 mg, or 300±15 mg, or 310±15 mg, or 340±15 mg, or 370±15 mg, or 400±15 mg, or 410±15 mg, or 440±15 mg, or 470±15 mg, or 500±15 mg, or 750±15 mg, or 1,000±15 mg.

In a preferred embodiment, particularly when the morphinan-derivative according to the invention is intended for administration over an extended period of time such as several months or years, it is preferred to initiate administration at a comparatively low daily dose and to consecutively, preferably steadily increase the daily dose over a titration period until the desired maximum daily dose has been reached (dose titration). Once the maximum daily dose has been reached, the titration period is terminated and continuous administration proceeds which may also include a subsequent reduction of the daily dose, if desired.

In the following embodiments, the daily dose of the morphinan-derivative is preferably administered on each day, independently of one another, all at once (once daily, sid), divided in two portions (twice daily, bid), divided in three portions (thrice daily), or divided in four portions (four times daily).

In a preferred embodiment, the morphinan-derivative is administered by injection twice daily, once daily or less frequently, e.g. once in a week, optionally in combination with other drugs, such as in combination with liraglutide, preferably once daily, or in combination with exendin-4, e.g. twice daily or only once in a week.

In a preferred embodiment, the titration regimen is biphasic, i.e. includes the administration of two different daily doses d₁ and d₂, wherein daily dose d₁ is administered during a first administration interval a₁, preferably on every day, and daily dose d₂ is administered during a second administration interval a₂, preferably on every day, which second administration interval a₂ follows the first administration interval a₁, and wherein daily dose d₁<daily dose d₂. Preferably, daily dose d₂ is the maximum daily dose to be finally administered, and daily dose d₁ is within the range of from 10 to 90 wt.-% of daily dose d₂, more preferably 20 to 80 wt.-%, still more preferably 30 to 70 wt.-%, and most preferably 40 to 60 wt.-% of daily dose d₂. Preferably, the first administration interval a₁ comprises at least 2 days, more preferably at least 4 days, still more preferably at least 7 days, yet more preferably at least 14 days, even more preferably at least 21 days, most preferably at least 28 days, and in particular at least 2 months. Preferably, the second administration interval a₂ comprises at least 2 days, more preferably at least 4 days, still more preferably at least 7 days, yet more preferably at least 14 days, even more preferably at least 21 days, most preferably at least 28 days, and in particular at least 2 months. Thus, according to this embodiment, the titration period comprises the first administration interval a₁.

In another preferred embodiment, the titration regimen is triphasic, i.e. includes the administration of three different daily doses d₁, d₂ and d₃, wherein daily dose d₁ is administered during a first administration interval a₁, preferably on every day, daily dose d₂ is administered during a second administration interval a₂, preferably on every day, which second administration interval a₂ follows the first administration interval a₁, and daily dose d₃ is administered during a third administration interval a₃, preferably on every day, which third administration interval as follows the second administration interval a₂, and wherein daily dose d₁<daily dose d₂<daily dose d₃. Preferably, daily dose d₃ is the maximum daily dose to be finally administered; and daily dose d₁ is within the range of from 5 to 55 wt.-% of daily dose d₃, more preferably 10 to 50 wt.-%, still more preferably 15 to 45 wt.-%, and most preferably 20 to 40 wt.-% of daily dose d₃; and daily dose d₂ is within the range of from 45 to 95 wt.-% of daily dose d₃, more preferably 50 to 90 wt.-%, still more preferably 55 to 85 wt.-%, and most preferably 60 to 80 wt.-% of daily dose d₃. Preferably, the first administration interval a₁ comprises at least 2 days, more preferably at least 4 days, still more preferably at least 7 days, yet more preferably at least 14 days, even more preferably at least 21 days, most preferably at least 28 days, and in particular at least 2 months. Preferably, the second administration interval a₂ comprises at least 2 days, more preferably at least 4 days, still more preferably at least 7 days, yet more preferably at least 14 days, even more preferably at least 21 days, most preferably at least 28 days, and in particular at least 2 months. Preferably, the third administration interval as comprises at least 2 days, more preferably at least 4 days, still more preferably at least 7 days, yet more preferably at least 14 days, even more preferably at least 21 days, most preferably at least 28 days, and in particular at least 2 months. Thus, according to this embodiment, the titration period comprises the first administration interval a₁ as well as the second administration interval a₂.

In a preferred embodiment, the titration regimen is multiphasic, i.e. includes the administration of a multitude of different daily doses d₁, d₂, d₃, . . . dₙ, wherein daily dose d₁ is administered during a first administration interval a₁, preferably on every day, daily dose d₂ is administered during a second administration interval a₂, preferably on every day, which second administration interval a₂ follows the first administration interval a₁, daily dose d₃ is administered during a third administration interval a₃, preferably on every day, which third administration interval as follows the second administration interval a₂, and so on, until daily dose dₙ is administered during a final administration interval an of the titration period, preferably on every day, and wherein daily dose d₁<daily dose d₂<daily dose d₃< . . . <dₙ. For example, daily dose d₁ may amount to 120 mg of the morphinan-derivative. Daily dose d₁ may be administered all at once (once daily, sid), divided in two portions each amounting to 60 mg (twice daily, bid), divided in three portions each amounting to 40 mg (thrice daily), or divided in four portions each amounting to 30 mg (four times daily). During the titration phase, the daily dose d₁ may be increased up to a maximum daily dose dₙ of e.g. 960 mg. For example, during a titration phase of four weeks the daily dose may be increased by 30 mg to 60 mg, e.g. every three days, unless the patient reports complete therapeutic effect, side effects that interfere with daily activities, or unless the maximum daily dose dₙ is reached. Thus, the further increase of the daily dose during the titration phase depends on the perception of the patient. In the following administration interval (maintenance phase), the highest well-tolerated daily dose can be maintained at a constant level.

The invention furthermore relates to medicaments comprising at least one morphinan-derivative according to the invention and/or physiologically acceptable salts and stereoisomers thereof, including mixtures thereof in all ratios, and at least one further medicament active ingredient.

The invention also relates to a set (kit) comprising separate packs of (a) an effective amount of a morphinan-derivative according to the invention and/or physiologically acceptable salts and stereoisomers thereof, including mixtures thereof in all ratios; and (b) an effective amount of a further medicament active ingredient.

The set comprises suitable containers, such as boxes, individual bottles, bags or ampoules. The set may, for example, comprise separate ampoules, each containing an effective amount of a compound according to the invention and/or physiologically acceptable salts and stereoisomers thereof, including mixtures thereof in all ratios, and an effective amount of a further active ingredient in dissolved or lyophilised form. Preferably, said further active ingredient is metformin or a physiologically acceptable salt thereof.

The morphinan-derivatives are suitable as pharmaceutical active ingredients for mammals, in particular for humans, in the treatment or prophylaxis of diabetes type 1, diabetes type 2; latent autoimmune diabetes in adults (LADA), obesity; neuropathy and/or nephropathy, stroke, ischemia-reperfusion (I/R) injury, cardiovascular diseases (such as myocardial infarction and acute coronary syndrome), hypertension, preferably diabetic nephropathy and neuropathy as well as I/R injury; and cancer.

The invention thus relates to the use of morphinan-derivatives and to physiologically acceptable derivatives, solvates and stereoisomers, including mixtures thereof in all ratios, for the preparation of a medicament for the treatment or prophylaxis of diabetes type 1, diabetes type 2; obesity; neuropathy and/or nephropathy, preferably diabetic neuropathy, I/R injury; and cancer.

The morphinan-derivatives of the invention can be used as prophylactics or therapeutic agents for treating diseases or disorders mediated by deficient levels of GLP-1 activity or which can be treated by activating TGR5 including, but not limited to, diabetes mellitus, impaired glucose tolerance (IGT), impaired fasting glucose (IFG) and elevated levels of glycated hemoglobin (HbAlc) as well as other diseases and disorders such as those discussed below. Furthermore, the morphinan-derivatives of the invention can be also used to prevent the progression of the borderline type, IGT and IFG to diabetes mellitus.

The morphinan-derivatives of the invention can be also used as prophylactics or therapeutic agents of diabetic complications such as, but not limited to, neuropathy, nephropathy, preferably diabetic neuropathy, retinopathy, cataract, macroangiopathy, cerebrovascular disease (e.g. stroke), cardiovascular disease (e.g. acute coronary syndrome, myocardial infarction, heart failure), I/R injury, endothelial dysfunction, hypertension, diabetic foot, osteopenia, diabetic hyperosmolar coma, infectious diseases (e.g., respiratory infection, urinary tract infection, gastrointestinal tract infection, dermal soft tissue infection, lower limb infection etc.), diabetic gangrene, xerostomia, decreased sense of hearing, peripheral circulatory disturbance, cancer, etc.

The morphinan-derivatives of the invention can be also used as prophylactics or therapeutic agents in the treatment of diseases and disorders such as, but not limited to, obesity, metabolic syndrome (syndrome X), hypertension, hyperinsulinemia, hypoinsulinemia, hyperinsulinemia-induced sensory disorder, hypoinsulinemia-induced sensory disorder, dyslipoproteinemia (abnormal lipoproteins in the blood) including diabetic dyslipidemia, hyperlipidemia, hyperlipoproteinemia (excess of lipoproteins in the blood) including type I, II-a (hypercholesterolemia), II-b, III, IV (hypertriglyceridemia) and V (hypertriglyceridemia), low HDL levels, high LDL levels, atherosclerosis and its sequelae, vascular restenosis, neurodegenerative disease, depression, CNS disorders, liver steatosis, osteoporosis, hypertension, renal diseases (e.g., diabetic nephropathy, glomerular nephritis, glomeruloscierosis, nephrotic syndrome, hypertensive nephrosclerosis, terminal renal disorder etc.), cardiovascular disease (e.g. myocardial infarction, heart failure), angina pectoris, and cerebrovascular disease (e.g., cerebral infarction, cerebral apoplexy).

The morphinan-derivatives of the invention can also be used as prophylactics or therapeutic agents in the treatment of diseases and disorders such as, but not limited to, osteoporosis, fatty liver, hypertension, insulin resistant syndrome, inflammatory diseases (e.g., chronic rheumatoid arthritis, spondylitis deformans, osteoarthritis, lumbago, gout, postoperative or traumatic inflammation, remission of swelling, neuralgia, pharyngolaryngitis, cystitis, hepatitis (including non-alcoholic steatohepatitis), pneumonia, inflammatory colitis, ulcerative colitis), pancreatitis, visceral obesity syndrome, cachexia (e.g., carcinomatous cachexia, tuberculous cachexia, diabetic cachexia, hemopathic cachexia, endocrinopathic cachexia, infectious cachexia, cachexia induced by acquired immunodeficiency syndrome), polycystic ovary syndrome, muscular dystrophy, tumor (e.g., leukemia, breast cancer, prostate cancer, skin cancer etc.), irritable bowel syndrome, acute or chronic diarrhea, spondylitis deformans, osteoarthritis, remission of swelling, neuralgia, pharyngolaryngitis, cystitis, sudden infant death syndrome (SIDS), and the like.

The morphinan-derivatives of the invention can be used in combination with one or more additional drugs such as described below. The dose of the second drug can be appropriately selected based on a clinically employed dose. The proportion of the morphinan-derivatives and the second drug can be appropriately determined according to the administration subject, the administration route, the target disease, the clinical condition, the combination, and other factors. In cases where the administration subject is a human, for instance, the second drug may be used in an amount of 0.01 to 100 parts by weight per part by weight of the morphinan-derivatives.

The second compound of the pharmaceutical combination, pharmaceutical composition or dosing regimen preferably has complementary activities to the morphinan-derivative such that they do not adversely affect each other. Such drugs are suitably present in combination in amounts that are effective for the purpose intended. Accordingly, another aspect of the invention provides a composition comprising a morphinan-derivative according to the invention, or a solvate, metabolite, or pharmaceutically acceptable salt or prodrug thereof, in combination with a second drug, such as described herein.

The morphinan-derivative and the additional pharmaceutically active agent(s) may be administered together in a unitary pharmaceutical composition or separately and, when administered separately this may occur simultaneously or sequentially in any order. Such sequential administration may be close in time or remote in time. The amounts of the morphinan-derivative and the second agent(s) and the relative timings of administration will be selected in order to achieve the desired combined therapeutic effect.

The combination therapy may provide “synergy” and prove “synergistic”, i.e., the effect achieved when the active ingredients used together is greater than the sum of the effects that results from using the compounds separately. A synergistic effect may be attained when the active ingredients are (1) co-formulated and administered or delivered simultaneously in a combined, unit dosage pharmaceutical composition; (2) delivered by alternation or in parallel as separate pharmaceutical compositions; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The morphinan-derivatives of the invention can be used, for example in combination with additional drug(s) such as a therapeutic agent for diabetes mellitus, and/or a therapeutic agent for diabetic complications, as defined above.

Examples of known therapeutic agents for diabetes mellitus which can be used in combination with a morphinan-derivative include insulin preparations (e.g., animal insulin preparations extracted from the bovine or swine pancreas; human insulin preparations synthesized by a genetic engineering technique using Escherichia coli or a yeast), a fragment of insulin or derivatives thereof (e.g., INS-i), agents for improving insulin sensitivity (e.g., pioglitazone hydrochloride, troglitazone, rosiglitazone or its maleate, GI-262570, JTT-50 1, MCC-555, YM-440, KRP-297, CS-Oil, FK-614), alpha-glucosidase inhibitors (e.g., voglibose, acarbose, miglitol, emiglitate), biguanides (e.g., phenformin, metformin, buformin), insulin secretagogues [sulfonylureas (e.g., tolbutamide, glibenclamide, gliclazide, chiorpropamide, tolazamide, acetohexamide, glyclopyramide, glimepiride, glipizide, glybuzole), repaglinide, nateglinide, mitiglinide or its calcium salt hydrate, GLP-1J, exendin-4, liraglutide and other incretinbased drugs or coagonists, dipeptidylpeptidase IV inhibitors (e.g., NVP-DPP-278, PT-100, sitagliptin, vildagliptin), beta-3 agonists (e.g., CL-3 16243, SR-58611-A, UL-TG-307, SB-226552, AJ-9677, BMS196085, AZ-40140 etc.), amylin agonists (e.g., pramlintide), phosphotyrosine phosphatase inhibitors (e.g., vanadic acid), gluconeogenesis inhibitors (e.g., glycogen phosphorylase inhibitors, glucose-6-phosphatase inhibitors, glucagon antagonists), SGLT (sodium-glucose cotransporter) inhibitors (e.g., T1095, canagliflozin, dapagliflozin, empagliflozin), and the like.

Examples of known therapeutic agents for diabetic complications include aldose reductase inhibitors (e.g., tolrestat, epairestat, zenarestat, zopobestat, minairestat, fidarestat (SNK-860), CT-i 12), neurotrophic factors (e.g., NGF, NT-3, BDNF), neurotrophic factor production secretion promoters, PKC inhibitors (e.g., LY-333531), AGE inhibitors (e.g., ALT946, pimagedine, pyratoxathine, Nphenacylthiazolium bromide (ALT766), EXO-226), active oxygen scavengers (e.g., thioctic acid), cerebral vasodilators (e.g., tiapuride, mexiletine), anti-convulsants (e.g. pregabalin, gabapentin), anti-depressants (e.g. duloxetine, Nortripylin, desipramine), or opiates (e.g., tramadol, tapentadol).

The morphinan-derivatives of the invention can also be used, for example in combination with antihyperlipidemic agents. Epidemiological evidence has firmly established hyperlipidemia as a primary risk factor in causing cardiovascular disease (CVD) due to atherosclerosis. In recent years, emphasis has been placed on lowering plasma cholesterol levels, and low density lipoprotein cholesterol in particular, as an essential step in prevention of CVD.

Cardiovascular disease is especially prevalent among diabetic subjects, at least in part because of the existence of multiple independent risk factors in this population. Successful treatment of hyperlipidemia in the general population, and in diabetic subjects in particular, is therefore of exceptional medical importance. Examples of antihyperlipidemic agents include statin compounds which are cholesterol synthesis inhibitors (e.g., cerivastatin, pravastatin, simvastatin, lovastatin, atorvastatin, fluvastatin, itavastatin or their salts, etc.), squalene synthase inhibitors or fibrate compounds (e.g., bezafibrate, clofibrate, simfibrate, clinofibrate) having a triglyceride lowering action and the like.

The morphinan-derivatives of the invention can also be used, for example in combination with hypotensive or antithrombotic agents. Hypertension has been associated with elevated blood insulin levels, a condition known as hyperinsulinemia. Insulin, a peptide hormone whose primary actions are to promote glucose utilization, protein synthesis and the formation and storage of neutral lipids, also acts to promote vascular cell growth and increase renal sodium retention, among other things. These latter functions can be accomplished without affecting glucose levels and are known causes of hypertension.

Peripheral vasculature growth, for example, can cause constriction of peripheral capillaries, while sodium retention increases blood volume. Thus, the lowering of insulin levels in hyperinsulinemics can prevent abnormal vascular growth and renal sodium retention caused by high insulin levels and thereby alleviates hypertension. Examples of hypotensive agents include angiotensin converting enzyme inhibitors (e.g., captopril, enalapril, delapril), angiotensin II antagonists (e.g., candesartan cilexetil, losartan, eprosartan, valsantan, termisartan, irbesartan, tasosartan), calcium antagonists (e.g., manidipine, nifedipine, nicardipine, amlodipine, efonidipine), and clonidine. Examples for antithrombotic drugs include aspirin, clopidogrel, prasugrel, ticagrelor, cangrelor, eptifibatide, tirofiban, unfractioned heparin, enoxaparin, bivalirudin, fondaparinux, rivaroxaban, and dual antiplatelet therapy (DAPT) with aspirin and either ticagrelor or prasugrel (or another P2Y12 receptor inhibitor).

The morphinan-derivatives of the invention can be used in combination with anti-obesity agents. The term “obesity” implies an excess of adipose tissue. Obesity is a well-known risk factor for the development of many very common diseases such as diabetes, atherosclerosis, and hypertension. To some extent appetite is controlled by discrete areas in the hypothalamus: a feeding centre in the ventrolateral nucleus of the hypothalamus (VLH) and a satiety centre in the ventromedial hypothalamus (VMH). The cerebral cortex receives positive signals from the feeding center that stimulates eating, and the satiety center modulates this process by sending inhibitory impulses to the feeding center. Several regulatory processes may influence these hypothalamic centers. The satiety center may be activated by increases in plasma glucose and/or insulin that follow a meal. Examples of anti-obesity agents include anti-obesity drugs acting on the central nervous system (e.g., dexfenfluramine, fenfluramine, phentermine, sibutramine, anfepramon, dexamphetamine, mazindol, phenylpropanolamine, clobenzorex), pancreatic lipase inhibitors (e.g. orlistat), beta-3 agonists (e.g., CL-3 16243, SR-5861 1-A, UL-TG-307, SB-226552, AJ-9677, BMS-196085, AZ-40140), anorectic peptides (e.g., leptin, CNTF (Ciliary Neurotrophic Factor) and cholecystokinin agonists (e.g. lintitript, FPL-1 5849) as well as dual GIP and GLP-1 receptor agonists (e.g., tirzepatide) or glucagon and GLP-1 receptor coagonists (e.g., efinopegdutide, MED10382, cotadutide) ortriagonists (e.g., XFL6 or SAR441255).

The morphinan-derivatives of the invention can be used in combination with anti-cancer agents. These include, but are not limited to,
alkylating agents (e.g. cyclophosphamide, temozolomide, cisplatin),
antimetabolites (e.g., 5-fluorouracil, capecitabine, cytarabine, gemcitabine, hydroxyurea, methotrexate, pemetrexed),
anti-tumor antibiotics (e.g., doxorubicine, actinomycin-D, mitomycin-C),
topoisomerase inhibitors (e.g., topotecan, irinotecan, etoposide, teniposide, mitoxantrone),
mitotic inhibitors (e. g., paclitaxel, docetaxel, ixabepilone, vinblastine, vincristine, vinorelbine, estramustine),
tyrosine kinase inhibitors (e.g., imatinib, erlotinib),
corticosteroids (e.g., prednisone, methylprednisolone, dexamethasone),
L-asparaginase,
bortezomib, and
anti-angiogenesis drugs (e.g., bevacizumab).

For breast cancer treatment, the morphinan-derivatives are preferably combined with
surgery,
radiation therapy,
chemotherapy (e.g. with docetaxel, paclitaxel, cisplatin, carboplatin, vinorelbine, capecitabine, liposomal doxorubicin, gemcitabine, mitoxantrone, ixabepilone, albumin-bound paclitaxel or eribulin),
hormone therapy (e.g. toremifene, fulvestrant, letrozole, anastrozole or exemestane), targeted therapy (e.g. trastuzumab, pertuzumab, ado-trastuzumab emtansine or lapatinib), and/or
bone-directed therapy.

For pancreatic cancer treatment, the morphinan-derivatives are preferably combined with
surgery,
chemotherapy (e.g. gemcitabine, 5-FU, albumin-bound paclitaxel, erlotinib, capecitabine, leucovorin, irinotecan, oxaliplatin, cisplatin, paclitaxel, docetaxel, irinotecan liposome, doxorubicin, decarbazine, temozolomide, streptozocin, thalidomide),
radiation therapy,
hormone therapy (e.g. octreotide), and/or
targeted therapy (e.g erlotinib, sunitinib or everolimus).

For glioma treatment, the morphinan-derivatives are preferably combined with
surgery,
radiation therapy, and/or
chemotherapy (e.g. carmustine, temozolomide, procarbazine, lomustine or vincristine).

For ovarian cancer, the morphinan-derivatives are preferably combined with
surgery,
chemotherapy (e.g. cisplatin, carboplatin, paclitaxel, albumin-bound paclitaxel, altretamine, capecitabine, cyclophosphamide, etoposide, gemcitabine, ifosfamide, irinotecan, liposomal doxorubicin, melphalan, pemetrexed, tropotecan, vintorelbine or docetaxel),
radiation therapy,
hormone therapy (e.g. goserelin, leuprolide, aromatase, letrozole, anastrozole or exemestane), and/or
targeted therapy (e.g. bevacizumab or olaparib).

### EXAMPLES

The following examples further illustrate the invention, but are not to be construed as limiting its scope.

### Example 1

### Synthesis of (1S,9S)-4-methoxy-17-methyl-17-azatetracyclo[7.5.3.0¹,¹⁰,0²,⁷]heptadeca2(7),3,5-trien-5-amine dihydrochloride (3)

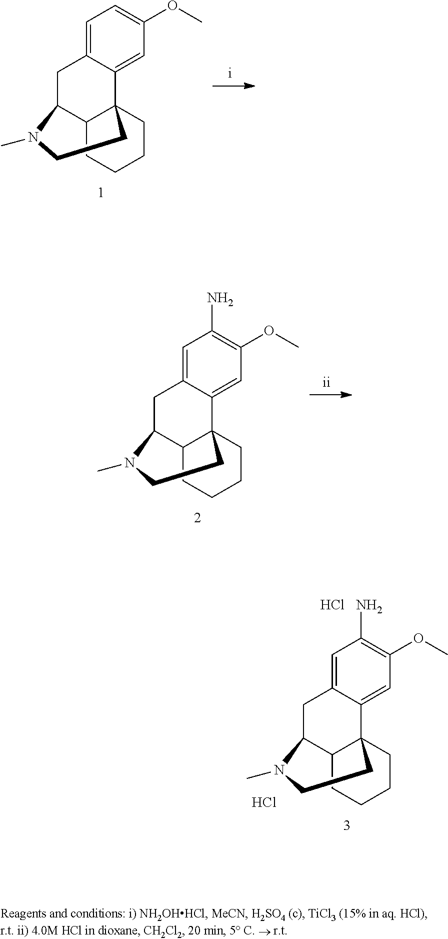

Dextromethorphan (1) (25.0 g, 0.092 mol) and hydroxylamine hydrochloride (19.4 g, 0.27 mol) were suspended in dry acetonitrile (300 mL) under nitrogen atmosphere. Then concentrated sulfuric acid (50.0 mL, 92.0 g, 0.92 mol) was added dropwise while the temperature of the reaction mixture was maintained approx. 25° C. (by water bath). Afterwards, TiCl₃ (15% in aqueous HCl) was added via a peristaltic pump (addition rate of 35 mL/h) until the purple colour of the TiCl₃ solution persisted for more than 10 min—in total 415 mL. During the addition, the reaction temperature was maintained below 30° C. The progress of the reaction was then monitored by LC-MS and after completion 1 M aqueous sodium citrate (100 mL) was added. Afterwards, the reaction mixture was cooled down to 0° C. and sodium hydroxide (as solid) was added portion-wise until the pH reached >7. Upon alkalization, TiO₂ precipitated and the obtained suspension was centrifuged. After decanting off the mother liquor, the solid was washed with chloroform/isopropanol mixture (7:3, 3×200 mL)—each wash cycle consisted of suspending the solid in the solvent mixture, centrifugation and decanting off the mother liquor. The combined mother liquors were concentrated under reduced pressure. The crude product (2) was filtered through silica gel (6 g silica gel/1 g crude), eluting with a mixture of dichloromethane/methanol/7 N NH₃ in methanol 100:2:1. The fractions containing product were combined and concentrated in vacuo. The isolated crude (2) was then dissolved in dichloromethane (50 mL) and the resulting solution was cooled down to 0° C. 4 N HCl in 1,4-dioxane (4 mol equivalents) was then added dropwise to the solution and the reaction mixture was left at room temperature for additional 30 min. Afterwards, the solvent was removed under reduced pressure. Final digestion of the solid with ethyl acetate/methanol 10:1 (25 mL/1 g crude) at room temperature and under sonication provided (1S,9S)-4-methoxy-17-methyl-17-azatetracyclo[7.5.3.0¹,¹⁰,0²,⁷]heptadeca-2(7),3,5-trien-5-amine dihydrochloride salt (3) as a white-off solid (14 g, >95% purity by uHPLC, 53% yield). The product is hygroscopic and turned to a brown oil after prolonged storage at room temperature.

¹H-NMR (300 MHz, CD₃OD): δ 7.27 (s, 1H), 7.14 (s, 1H), 3.97 (s, 3H), 3.68 (s, 2H), 3.24 (brs, 1H), 3.20 (d, J=5.6 Hz, 1H), 2.93 (s, 3H), 2.72-2.54 (m, 2H), 2.08 (dt, J=12.1, 2.8 Hz, 1H), 2.02 (s, 3H), 1.75-1.21 (m, 10H), 1.14 (dd, J=12.8, 3.8 Hz, 1H), 1.05 (dd, J=12.7, 3.8 Hz, 1H), MS (m/z): [M+H]⁺ calcd. for C₁₈H₂₇N₂O, 287.2; found, 287.2.

### Example 2

### Synthesis of N-[(1S,9S)-4-methoxy-17-methyl-17-azatetracyclo[7.5.3.0¹,¹⁰,0²,⁷]heptadeca2(7),3,5-trien-5-yl]-1-methyl-1H-imidazole-5-carboxamide dihydrochloride (5, cmp. 7, Kom56)

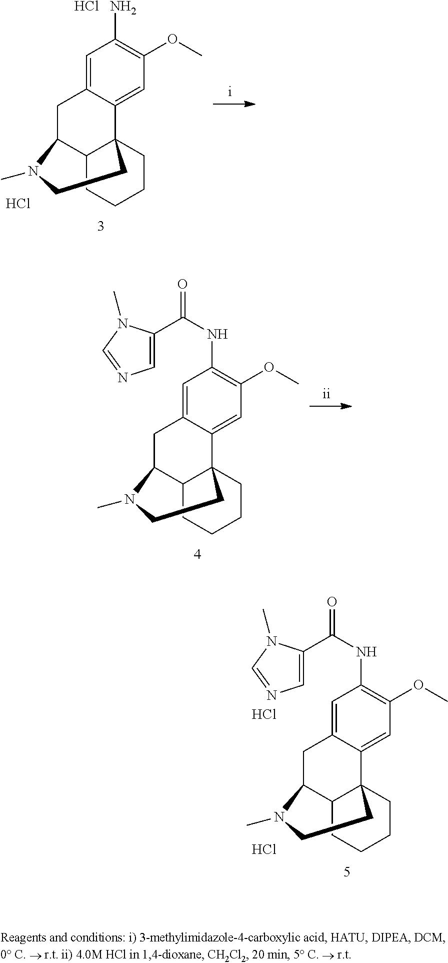

To a solution of 3 (26.1 g, 72.0 mmol) and 1-methyl-1H-imidazole-5-carboxylic acid (10.1 g, 79.8 mmol) in DCM (400 mL) at 0° C., were added HATU (31.2 g, 79.8 mmol) and DIPEA (38.0 g, 51 mL, 290.0 mmol). The reaction mixture was warmed up to room temperature and left at this temperature. The progress of the reaction was monitored by LC-MS and after full conversion was reached, the reaction mixture was washed with 1M aqueous NaOH (2×500 mL) and water (500 mL), dried over MgSO₄ and concentrated in vacuo. The isolated crude oil (4) was dissolved in dichloromethane (300 mL) and the resulting solution was cooled down to 0° C. Then 4.0 N HCl solution in 1,4-dioxane (4 mol equivalents) were slowly added to the mixture. The reaction mixture was allowed to warm up to room temperature and left at this temperature for further 20 min. The solvent was then removed under reduced pressure and the excess of hydrogen chloride was removed by co-evaporation with chloroform (3×50 mL). The crude product (5) was dissolved in dichloromethane/methanol 10:1, the solution was added to tertbutyl methyl ether (1.2 L) and the formed suspension was sonicated for 3 hours. The solid was filtered and washed with diethyl ether (3×100 mL). The obtained dried solid was suspended in tetrahydrofuran (1 L) and the suspension was heated to reflux. At this temperature, methanol was added slowly until the full amount of solid was dissolved. The solution was then cooled down to 0° C. and ethyl acetate (1 L), followed by diethyl ether (200 mL) were added to precipitate the solid. The suspension was further sonicated for 30 min and then filtered, washed with diethyl ether (3×100 mL) and finally dried in vacuo. The target N-[(1S,9S)-4-methoxy-17-methyl-17-azatetracyclo[7.5.3.0¹,¹⁰,0²,⁷]heptadeca-2(7),3,5-trien5-yl]-1-methyl-1H-imidazole-5-carboxamide dihydrochloride salt (5) was isolated as a white-off solid (20 g, >95% purity by ¹H-NMR, 70% yield).

¹H NMR (300 MHz, CD₃OD): δ 9.04 (s, 1H), 8.28 (s, 1H), 7.79 (s, 1H), 7.01 (s, 1H), 4.15 (s, 3H), 3.90 (s, 3H), 3.67 (brs, 1H), 3.27-3.04 (m, 4H), 2.93 (s, 3H), 2.75 (td, J=13.1, 3.3 Hz, 1H), 2.59 (d, J=13.9 Hz, 1H), 2.09 (dt, J=12.0, 3.0 Hz, 1H), 1.96 (td, J=13.8, 4.5 Hz, 1H), 1.76-1.13 (m, 9H); [M+H]⁺ calcd. for C₂₃H₃₁N₄O₂, 395.2; found, 395.2.

A skilled person recognizes that additional compounds according to the invention can be synthesized in analogy starting from (3).

### Further Synthesis Routes

### Synthesis of common intermediate (1S,9S)-5-iodo-4-methoxy-17-methyl-17-azatetracyclo[7.5.3.0¹,¹⁰,0²,⁷]heptadeca-2(7),3,5-triene (2) for final compounds 30-39 and 40, 42

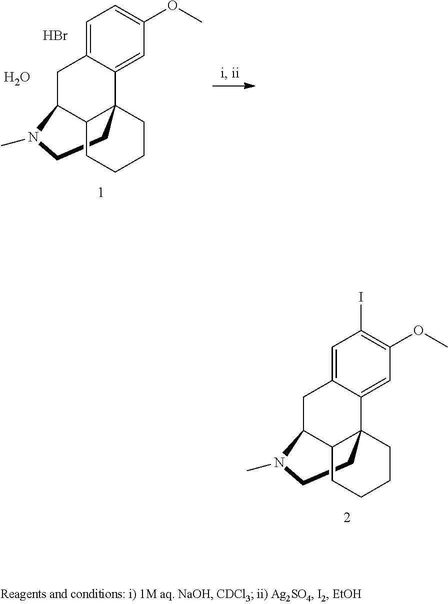

Commercially available dextromethorphan hydrobromide monohydrate (1) (50 g, 0.135 mol) was dissolved in chloroform (500 mL) and washed with 1M NaOH (200 mL). The organic phase was dried over magnesium sulfate and concentrated in vacuo. It was stored at −18° C. until needed.

To a solution of dextromethorphan as free base (10 g, 0.037 mol) in ethanol (270 mL) under nitrogen atmosphere was added silver sulfate (23 g, 0.074 mol) and iodine (18.7 g, 0.074 mol) and the reaction mixture was left at room temperature for 15 hours.

The reaction progress was monitored by LC-MS and after full conversion was observed, the reaction mixture was filtered, the salts were further washed with ethanol (50 mL) and the filtrate was concentrated in vacuo.

The residue was dissolved in ethyl acetate (250 mL) and washed with 7% aq. ammonium hydroxide (pH˜9) (100 mL), followed by 17% aq. sodium thiosulfate solution (75 mL). The organic phase was dried over magnesium sulfate and concentrated in vacuo to provide the title compound with purity >80% which was used in the next step without further purification (12 g, 82%).

¹H-NMR (300 MHz, CDCl₃): δ 7.51 (s, 1H), 6.68 (s, 1H), 3.83 (s, 3H), 3.68 (s, 2H), 2.93 (d, J=18.3 Hz, 1H), 2.81-2.79 (m, 1H), 2.55 (dd, J=12.5, 5.8 Hz, 1H), 2.48-2.42 (m, 1H), 2.39 (s, 3H), 2.33-2.29 (m, 1H), 2.06 (td, J=12.4, 3.1 Hz, 1H), 1.83 (dt, J=13.0, 5.7 Hz, 1H), 1.73 (dd, J=12.6, 4.8 Hz, 1H), 1.65-1.62 (m, 1H), 1.55-1.51 (m, 1H), 1.42-1.25 (m, 5H), 1.10 (td, J=12.8, 3.3 Hz, 1); MS (m/z): [M+H]⁺ calcd. for C₁₈H₂₅INO, 398.3; found, 398.0.

General procedure 1, synthesis of compounds 30-38 and 42:

### Synthesis of 1-[(1S,9S)-4-methoxy-17-methyl-17-azatetracyclo[7.5.3.0¹,¹⁰,0²,⁷]heptadeca2(7),3,5-trien-5-yl]-N,N-dimethylazetidin-3-amine (35)

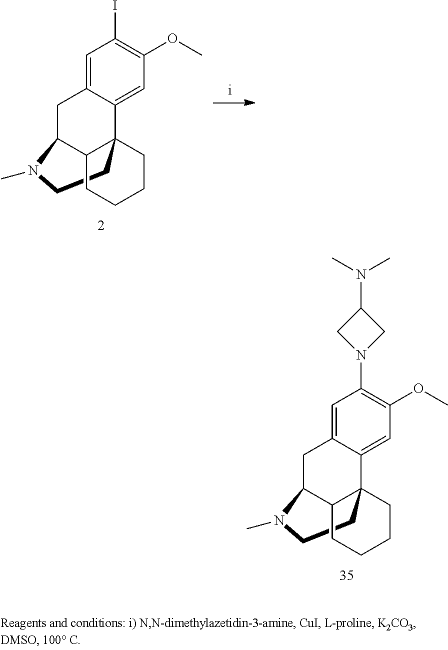

A solution of (1S,9S)-5-iodo-4-methoxy-17-methyl-17-azatetracyclo[7.5.3.0¹,¹⁰,0²,⁷]heptadeca2(7),3,5-triene 2 (1 g, 2.52 mmol), NN-dimethylazetidin-3-amine (872 mg, 5.02 mmol), CuI (144 mg, 0.76 mmol), L-proline (174 mg, 1.52 mmol) and pre-dried at 200° C. for 4 hours potassium carbonate (1.39 g, 10.07 mmol) in degassed dry DMSO (15 mL) under nitrogen atmosphere was heated to 100° C. for 20 hours.

The reaction progress was monitored by UHPLC and after completion, the reaction mixture was allowed to cool down to room temperature. It was then poured into water (150 mL) and extracted with chloroform/i-propanol 7:3 (2×100 mL). The combined organic extracts were dried over magnesium sulfate and concentrated in vacuo.

The crude was purified by flash column chromatography on reverse phase with ACN/H₂O+0.1% HCOOH as solvent system. The fraction was neutralized with saturated aq. NaHCO₃ until pH˜8 and extracted with chloroform/i-propanol 7:3 (2×50 mL). The combined extracts were dried over MgSO4 and concentrated in vacuo, providing the title compound with purity >95% (250 mg, 27%).

¹H-NMR (300 MHz, CD₃OD): 6.71 (s, 1H), 6.30 (s, 1H), 4.04 (td, J=6.7, 0.8 Hz, 2H), 3.75 (s, 3H), 3.59-3.54 (m, 2H), 3.20-3.12 (m, 1H), 2.96 (d, J=18.3 Hz, 1H), 2.83-2.81 (m, 1H), 2.63 (dd, J=12.8, 5.7 Hz, 1H), 2.47-2.42 (m, 1H), 2.39 (s, 3H), 2.20 (s, 6H), 2.13-2.08 (m, 1H), 1.81-1.60 (m, 3H), 1.55-1.52 (m, 1H), 1.42-1.29 (m, 6H), 1.22-1.13 (m, 1H); MS (m/z): [M+H]⁺ calcd. for C₂₃H₃₆N₃O, 370.5; found, 370.2.

General procedure 2, synthesis of compounds 39 and 40:

### Synthesis of (1S,9S)-4-methoxy-17-methyl-5-(1H-1,2,4-triazol-1-yl)-17-azatetracyclo[7.5.3.0¹,¹⁰,0²,⁷]heptadeca-2,4,6-triene (39)

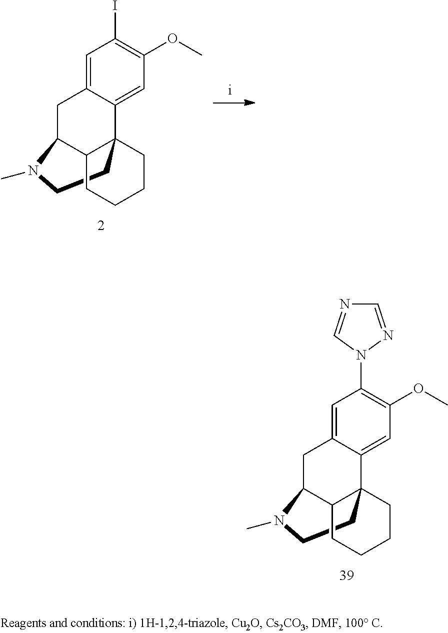

A solution of (1S,9S)-5-iodo-4-methoxy-17-methyl-17-azatetracyclo[7.5.3.0¹,¹⁰,0²,⁷]heptadeca2(7),3,5-triene 2 (200 mg, 0.5 mmol), 1H-1,2,4-triazole (104 mg, 1.5 mmol), Cu₂O (7 mg, 0.05 mmol) and Cs₂CO₃ (328 mg, 1.0 mmol) in degassed dry DMF (2 mL) under nitrogen atmosphere was heated to 100° C. for 14 hours.

The reaction progress was monitored by UHPLC and even though completion was not reached, impurities were starting to form, therefore reaction was stopped at ˜50% conversion.

The reaction mixture was allowed to cool down to room temperature and then was diluted with EtOAc (10 mL) and filtered. The filtrate was washed with water (20 mL) and the aqueous layer was further extracted with EtOAc (2×10 mL). The combined organic extracts were dried over magnesium sulfate and concentrated in vacuo.

The crude was purified by preparative HPLC with ACN/H₂O+0.1% HCOOH as solvent system. After lyophilization, the fraction was passed through ISOLUTE® SCX-2 cartridge—compound loaded as a solution in MeOH, washed with MeOH and lastly eluted with 2N NH₃ in MeOH, providing the title compound with purity ˜95% (4 mg, 2%).

¹H-NMR (300 MHz, CD₃OD): 8.91 (s, 1H), 8.14 (s, 1H), 7.53 (s, 1H), 7.13 (s, 1H), 3.92 (s, 3H), 3.19-3.13 (m, 1H), 2.88 (dd, J=19.0, 6.0 Hz, 1H), 2.75-2.71 (m, 1H), 2.58-2.54 (s, 3H overlapping with m, 1H), 2.35 (td, J=12.6, 3.1 Hz, 1H), 1.97-1.16 (m, 11H); MS (m/z): [M+H]⁺ calcd. for C₂₀H₂₇N₄₀, 339.4; found, 339.2.

General procedure 3, synthesis of compound 41:

### Synthesis of (1S,9S)-4-methoxy-17-methyl-5-(piperidin-1-yl)-17-azatetracyclo[7.5.3.0¹,¹⁰,0²,⁷]heptadeca-2,4,6-triene (41)

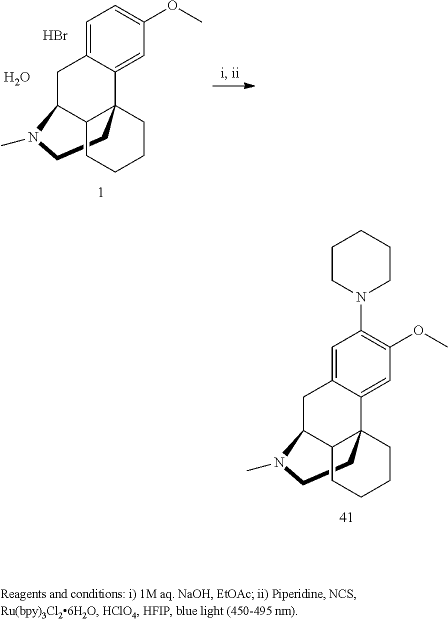

Commercially available dextromethorphan hydrobromide monohydrate (1) (50 g, 0.135 mol) was dissolved in EtOAc (500 mL) and washed with 1M NaOH (200 mL). The organic phase was dried over magnesium sulfate and concentrated in vacuo. It was stored at −18° C. until needed.

A solution of Ru(bpy)₃Cl₂·6H₂O (20 mg, 0.025 mmol), N-chlorosuccinimide (100 mg, 0.75 mmol), piperidine (75 μL, 0.75 mmol) in degassed hexafluoro-2-propanol (3 mL) was stirred for 60 min in the dark under nitrogen atmosphere.

Then, a solution of dextromethorphan as free base (135 mg, 0.45 mmol) and HClO4 (70% in water, 360 μL, 8.7 mmol) in hexafluoro-2-propanol (1 mL) were added to the reaction mixture. The reaction mixture was then placed immediately under blue light irradiation (450-495 nm).

The reaction progress was monitored by UHPLC and completion was observed 2 hours. The reaction mixture was quenched with saturated aq. NaHCO₃ (5 mL), then further diluted with brine (10 mL) and extracted with EtOAc (5×10 mL). The combined organic extracts were dried over magnesium sulfate and concentrated in vacuo.

The crude was purified by preparative HPLC with ACN/H₂O+0.1% HCOOH as solvent system. After lyophilization, the fraction was passed through ISOLUTE® SCX-2 cartridge—compound loaded as a solution in MeOH, washed with MeOH and lastly eluted with 2N NH₃ in MeOH, providing the title compound with purity >95% (64 mg, 36%).

¹H-NMR (300 MHz, CDCl₃): δ 6.64 (s, 1H), 6.60 (s, 1H), 3.80 (s, 3H), 3.03-2.88 (m, 5H), 2.83-2.81 (m, 1H), 2.57 (dd, J=18.1, 5.7 Hz, 1H), 2.47-2.43 (m, 1H), 2.41 (s, 3H), 2.32-2.28 (m, 1H), 2.12 (td, J=12.0, 3.1 Hz, 1H), 1.82 (dt, J=12.4, 2.9 Hz, 1H), 1.75-1.69 (m, 5H), 1.61-1.50 (m, 4H), 1.39-1.11 (m, 6H); MS (m/z): [M+H]⁺ calcd. for C₂₃H₃₅N₂O, 355.5; found, 355.2.

### Example 3

Compound 7 (5) (Kom56), dextromethorphan (1) (DXM) and (9S, 13S, 14S)-2-(1H-Imidazol1-yl)-3-methoxy-17-methyl morphinan dihydrobromide (DXM-2) were toxicologically tested using the SafetyScreen44™ Panel.

The results for the toxicology, inhibition of potassium voltage-gated channel subfamily H member 2 (hERG) and the μ-opioid (MOP) receptor are shown in the table here below:

| | 1 | | cmp. 7, 5 |
|---|---|---|---|
| | (DXM) | DXM-2 | Kom56 |
| toxicology (hits with over 50% inhibition) | 6 | 7 | 4 |
| hERG inhibition [%] | 20.9 | 51.8 | 7.9 |
| MOP inhibition [%] | 85 | 83.6 | 33.7 |

As demonstrated, compound 7 (5) (Kom56) exhibits less toxicology and significantly lower hERG inhibition and MOP inhibition than dextromethorphan (1) and (DXM-2).

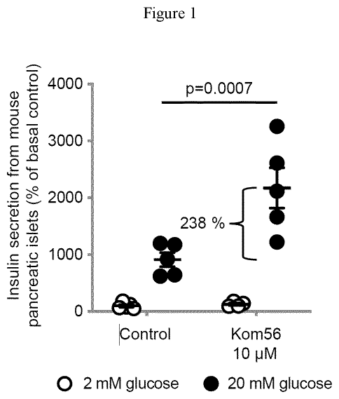

FIG. 1 shows experimental data demonstrating that compound 7 (5) (Kom56) increases the glucose-stimulated insulin secretion from mouse pancreatic islets: Insulin secretion from isolated mouse pancreatic islets under low (2 mM) and high glucose concentration (20 mM), and in the absence or presence of Kom56. n=4-5 islet batches each. Statistical testing: two-way ANOVA followed by Sidak's multiple comparison test. All values are shown with mean±SEM.

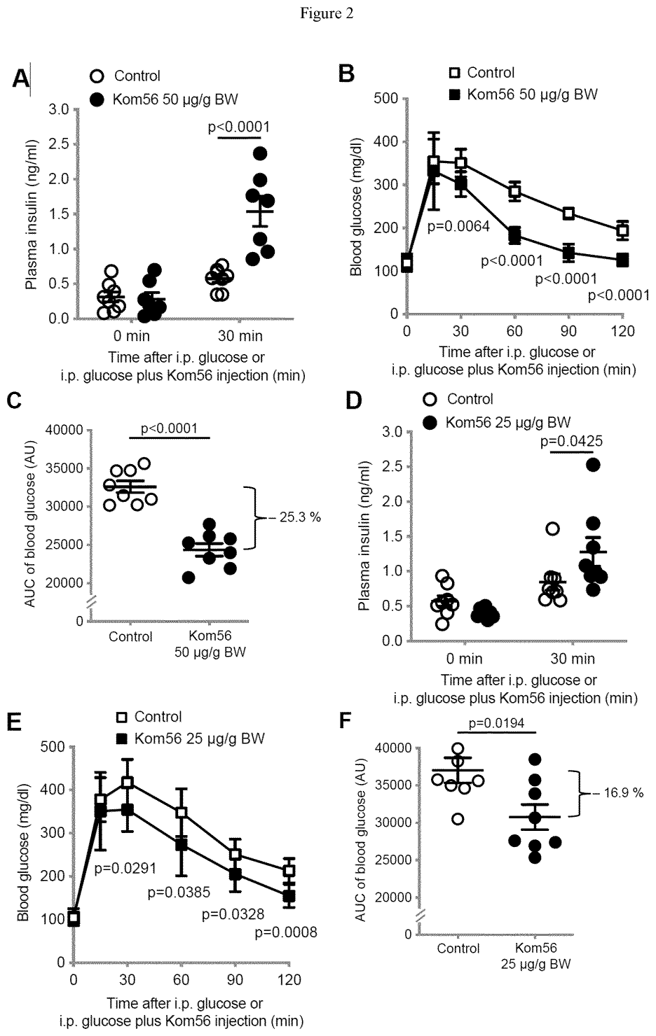

FIG. 2 shows experimental data demonstrating that compound 7 (5) (Kom56) increases plasma insulin concentrations and improves glucose tolerance after intraperitoneal administration: (A,D) In vivo plasma insulin concentrations before and 30 min after intraperitoneal (i.p.) compound administration together with glucose. (A) n=8 mice for control and n=7 mice for Kom56, (D) n=8 mice each. (B,E) Blood glucose concentrations of mice during i.p. glucose tolerance tests with 1.5 mg/g BW glucose with or without i.p. compound administration. n=8 mice each. (C,F) AUC₀₋₁₂₀ₘᵢₙ calculation of blood glucose concentrations of B,E. Statistical testing: (A,D) two-way ANOVA followed by Sidak's multiple comparison test. (B,C,E,F) unpaired two-sided Student's t-test. All values are shown with mean±SEM, except for glucose tolerance tests (B,E), in which all values are mean±SD.

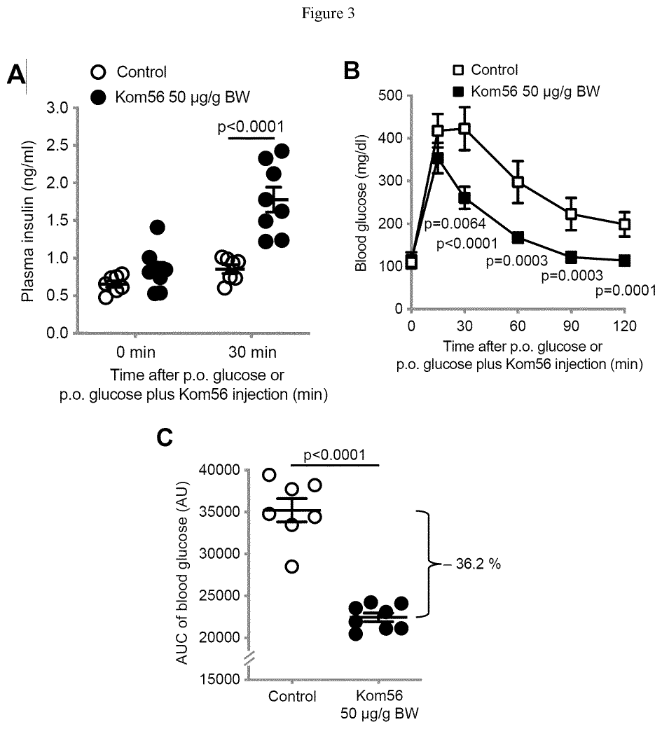

FIG. 3 shows experimental data demonstrating that compound 7 (5) (Kom56) increases plasma insulin concentrations and improves glucose tolerance after peroral administration: (A) In vivo plasma insulin concentrations before and 30 min after peroral (p.o.) compound administration together with glucose. n=7 mice for control and n=8 mice for Kom56. (B) Blood glucose concentrations of mice during oral glucose tolerance test with 1.5 mg/g BW glucose with or without p.o. compound administration. n=7 mice for control and n=8 mice for Kom56. (C) AUC₀₋₁₂₀ₘᵢₙ calculation of blood glucose concentrations of B. Statistical testing: (A) two-way ANOVA followed by Sidak's multiple comparison test. (B,C) unpaired two-sided Student's t-test. All values are shown with mean±SEM, except for glucose tolerance test (B), in which all values are mean±SD.

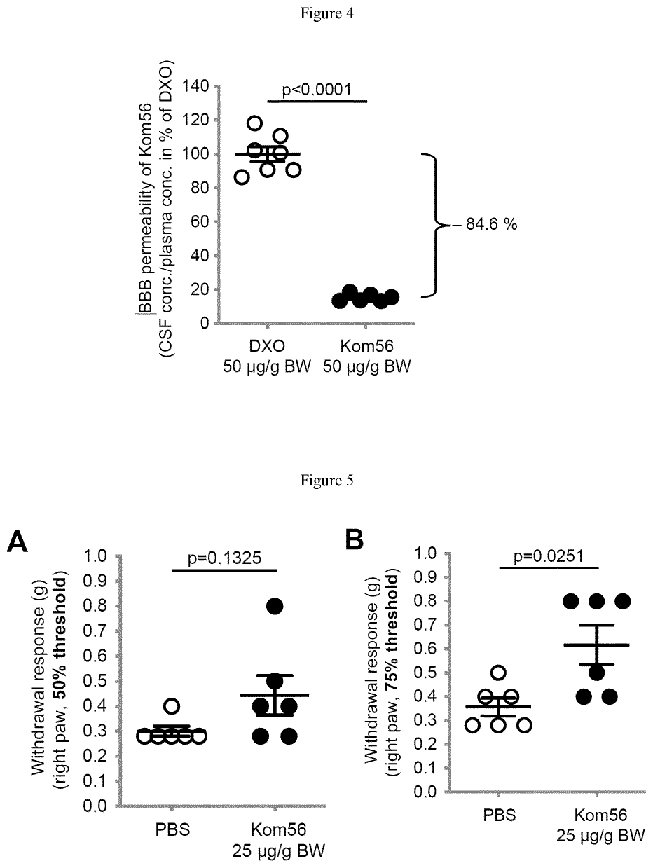

FIG. 4 shows experimental data demonstrating that compound 7 (5) (Kom56) displays a reduced blood-brain barrier (BBB) permeability: BBB permeability of Kom56 compared to DXO. Cerebrospinal fluid (CSF) was taken from the cisterna magna, while blood was taken from the heart to determine the concentrations of DXO and Kom56 by tandem mass spectrometry (MS/MS). CSF and blood samples were taken 30 min after intraperitoneal (i.p.) administration of compounds. n=7 mice for DXO and n=6 mice for Kom56. Statistical testing: unpaired two-sided Student's t-test. All values are shown with mean±SEM.

FIG. 5 shows experimental data demonstrating that compound 7 (5) (Kom56) reduces touch sensitivity during mechanical stimulation with Von Frey monofilaments: (A,B) Thresholds of paw withdrawal response (right paw) after intraperitoneal (i.p.) administration of PBS and Kom56. (A) Force that is needed to reach a 50% threshold, and (B) force that is needed to reach a 75% threshold in paw withdrawal. n=6 mice each. Statistical testing: (A,B) unpaired two-sided Student's t-test. All values are shown with mean±SEM.

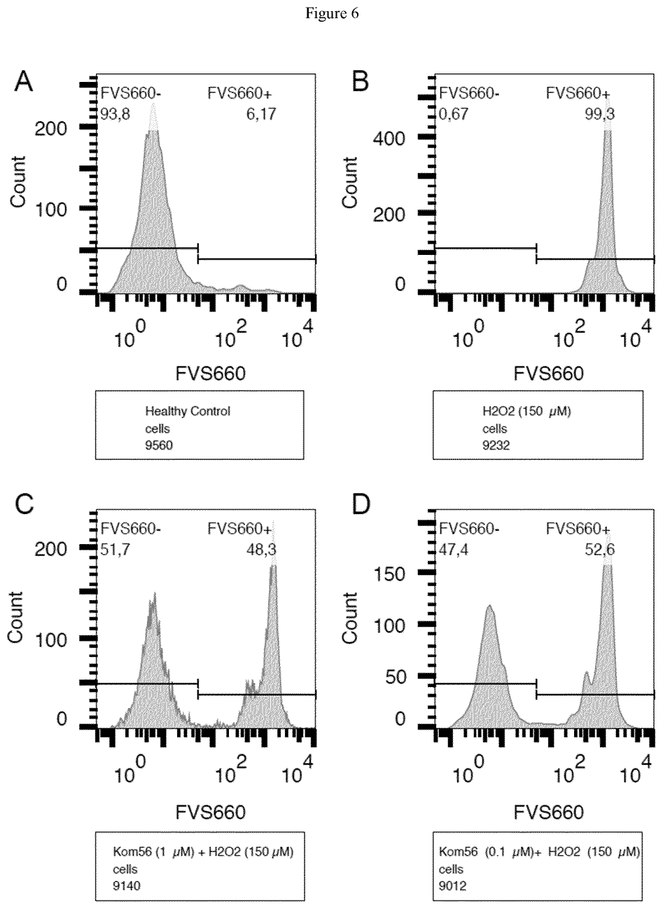

FIG. 6 shows experimental data demonstrating that compound 7 (5) (Kom56) protects mouse pancreatic islets from cell death induced by oxidative stress: (A-D) Mouse pancreatic islets incubated with or without 150 μM H₂O₂ either alone or in combination with 1 and 0.1 μM Kom56 for 24 h. (A) Healthy control, (B) pancreatic islets incubated with 150 μM H₂O₂, (C) 150 μM H₂O₂ plus 1 μM Kom56, and (D) 150 μM H₂O₂ plus 0.1 μM Kom56. Analyzed by flow cytometry. Pancreatic islets were dissociated using trypsin and stained with Fixable Viability Stain 660 (FVS660). FVS660+=dead cells, FVS660-=living cells, values are shown in %.

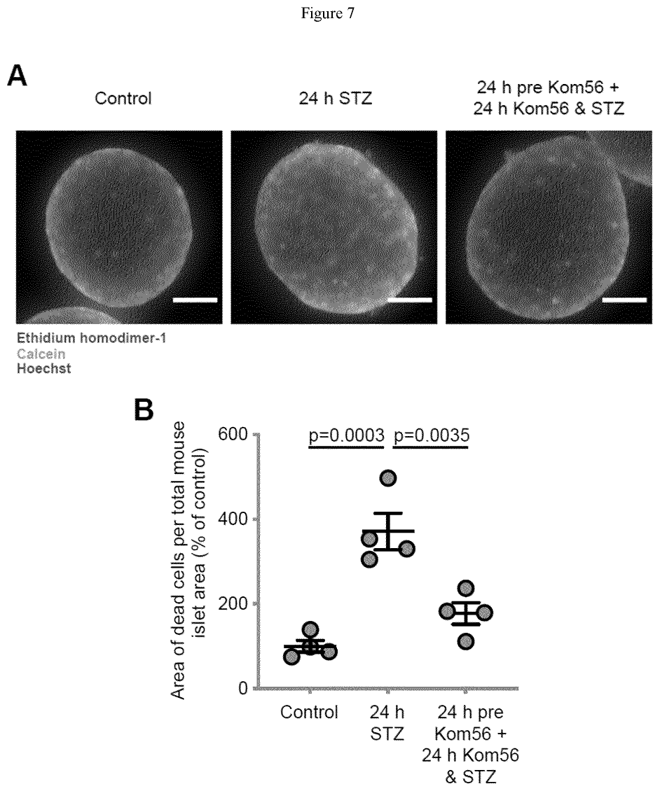

FIG. 7 shows experimental data demonstrating that compound 7 (5) (Kom56) protects mouse pancreatic islets from streptozotocin-induced cell death: (A) Representative Apotome images (maximum intensity projections) of mouse islets incubated with or without 2 mM streptozotocin (STZ) either alone or in combination with 1 μM Kom56 for 24 h. Blue (Hoechst), nuclei; red (ethidium homodimer1), dead cells; green (calcein), viable cells. (B) Area of dead cells in mouse islets treated as described in A. n=4 islet batches each with 11-14 islets per batch for each group. Statistical testing: (B) one-way ANOVA followed by Tukey's multiple comparison test. All values are shown with mean±SEM. (A) Scale bars, 50 μm.

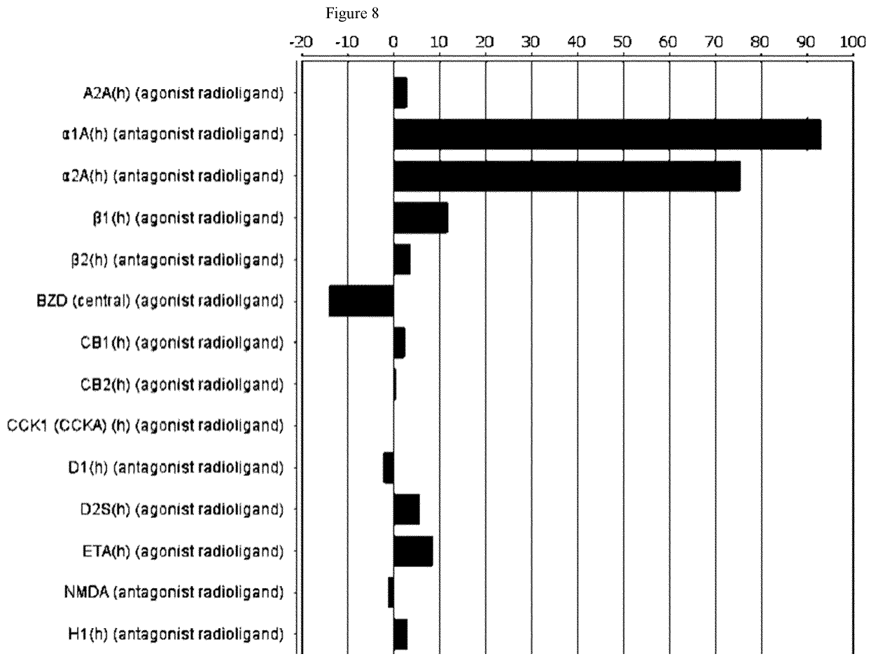

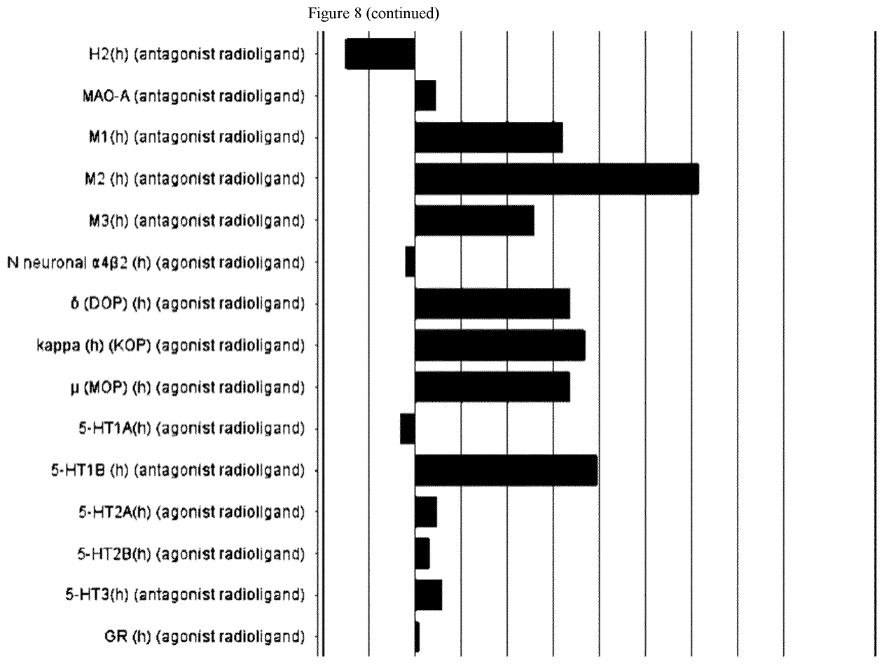

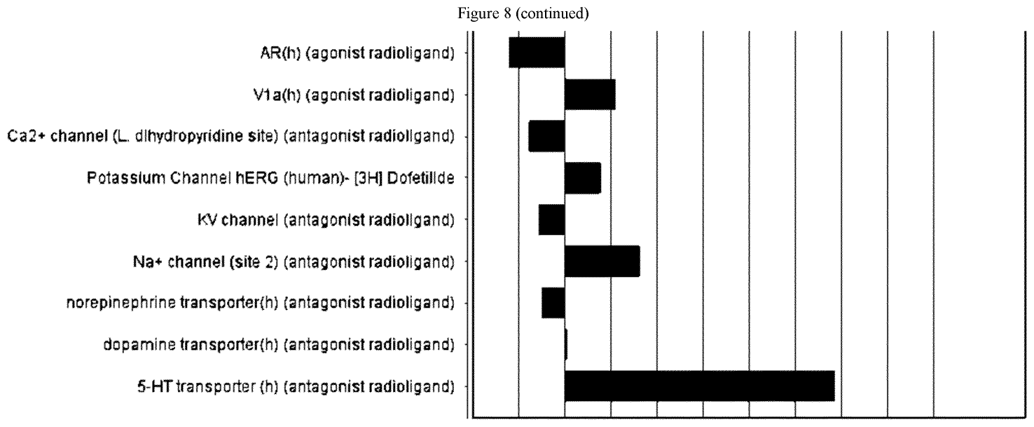

FIG. 8 shows a histogram for compound 7 (5) (Kom56) with regard to % inhibition of control specific binding (test concentration 1.0 10⁻⁵ M).

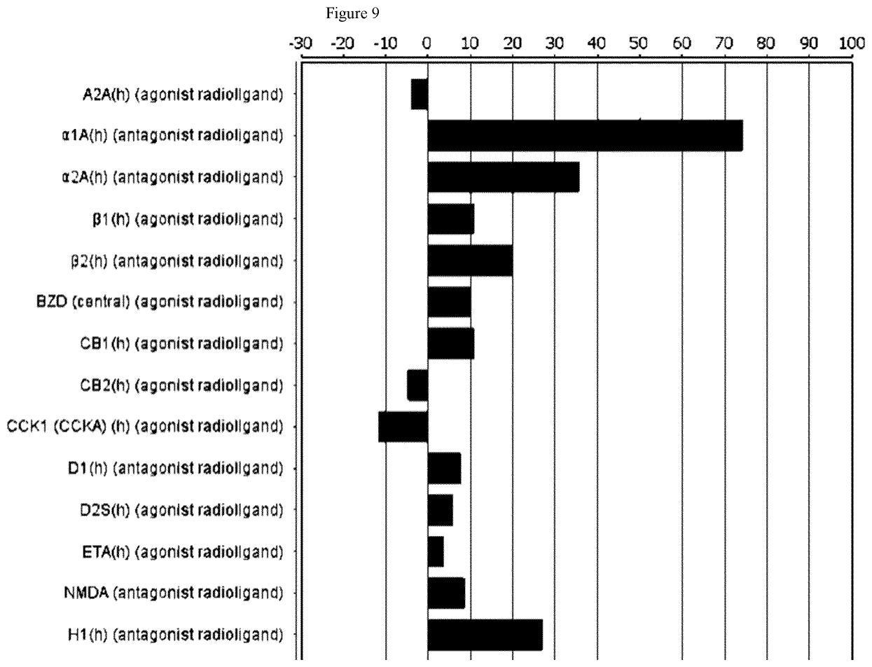

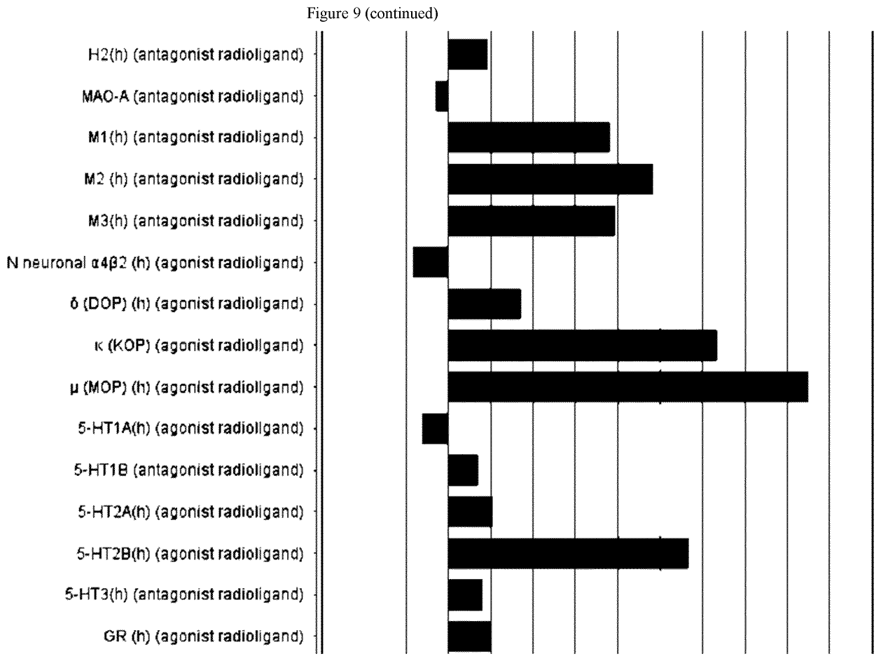

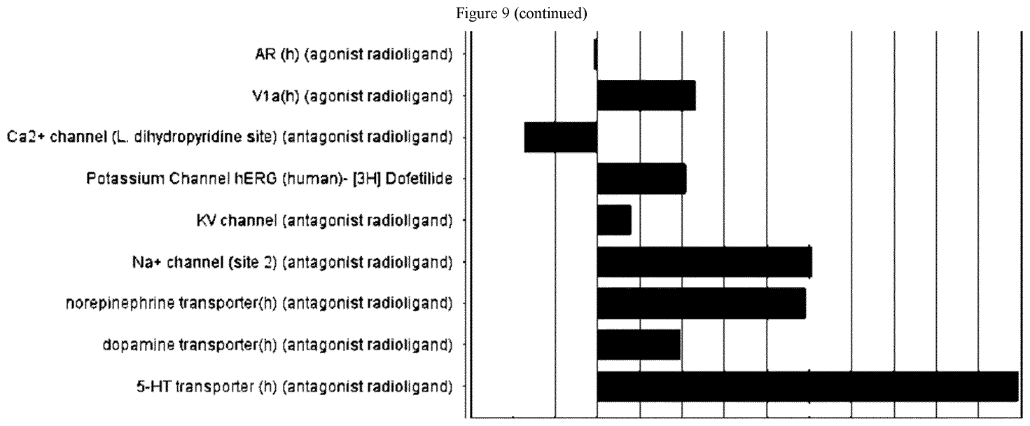

FIG. 9 shows a histogram for dextromethorphan (1) (DXM) with regard to % inhibition of control specific binding (test concentration 1.0 10⁻⁵ M).

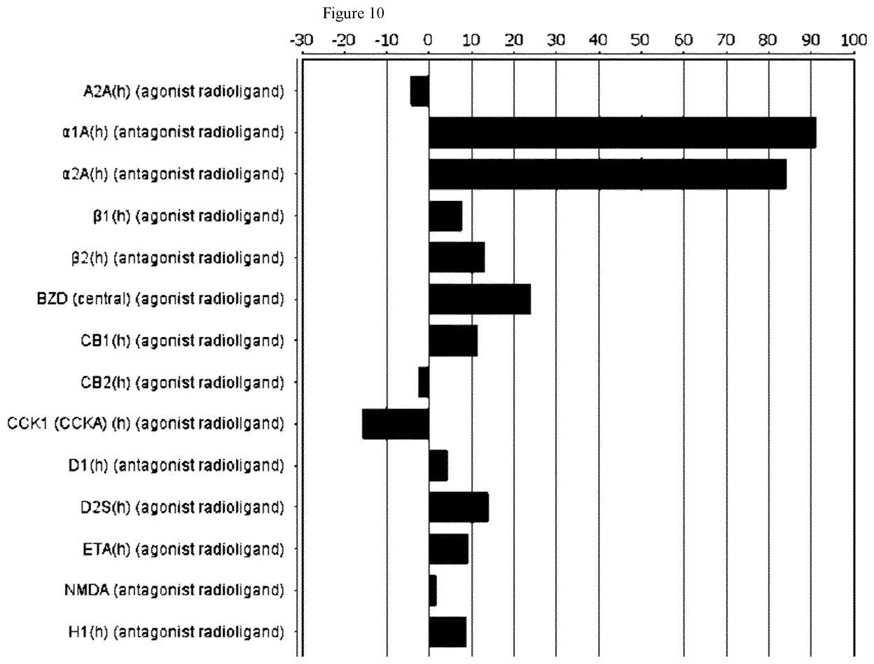

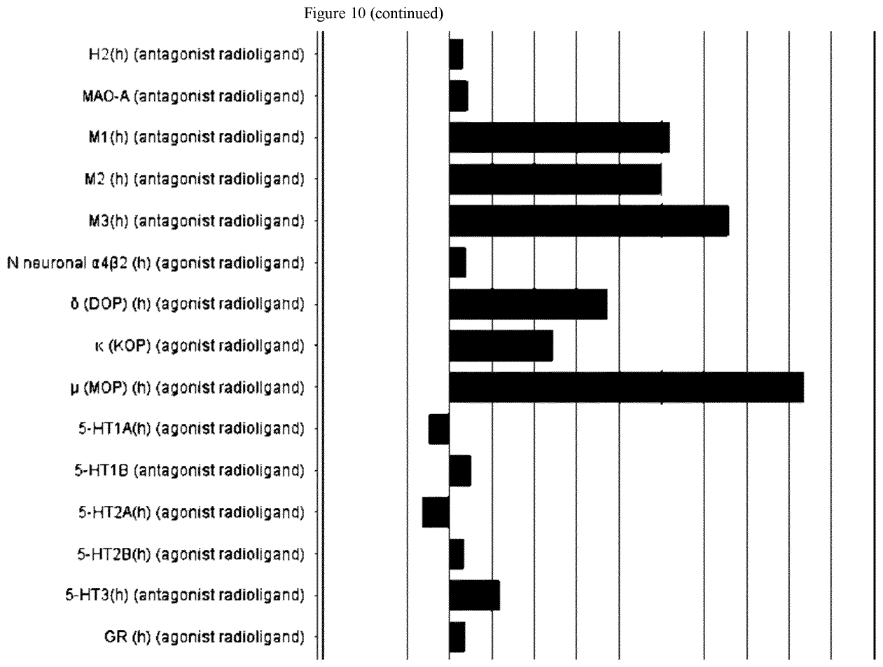

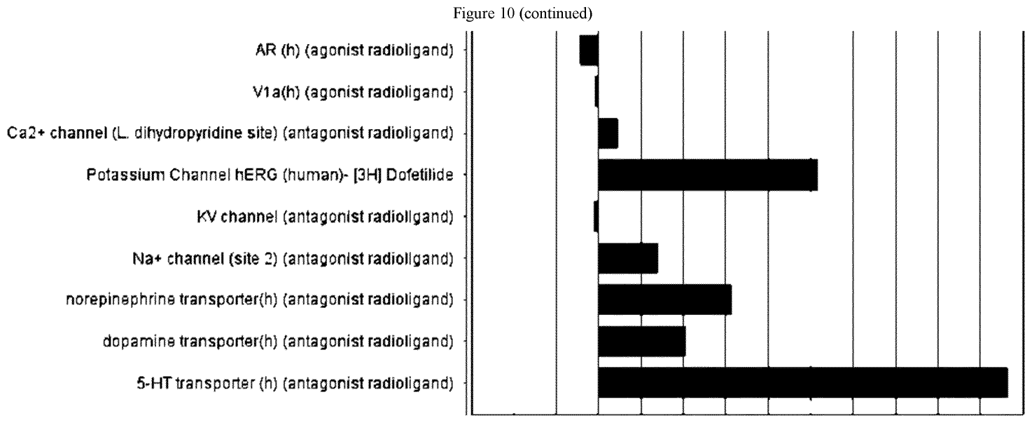

FIG. 10 shows a histogram for (9S, 13S, 14S)-2-(1H-Imidazol-1-yl)-3-methoxy-17-methyl morphinan dihydrobromide (DXM-2) with regard to % inhibition of control specific binding (test concentration 1.0 10⁻⁵ M).

### Example 4

Inhibition of potassium voltage-gated channel subfamily H member 2 (hERG) of the compounds was measured as in the SafetyScreen44™ Panel (see Example 3). The hERG channel binding assay is part of the SafetyScreen44™ Panel, but can also be conducted solely. The entire SafetyScreen44™ Panel with all 44 targets was just conducted for the three compounds as shown in [0206], for the other molecules just the hERG inhibition was measured.

For the hERG channel binding assay, compound binding was calculated as % inhibition of the binding of a radioactively labeled ligand that is specific for the target (i.e., [3H] Dofetilide). HEK-293 cells (human embryonic kidney cells) were used for these binding assays. The compounds were tested at a concentration of 10 μM and 2 repetitions were conducted for each compound.

Similar to compound 7, compounds 3, 5, 9, 15, 17, 22, 27, 31, 35, 36 and 37 exhibited a hERG inhibition of <15% (% inhibition of the binding of a ligand specific for the target).

### Example 5

Further, insulin secretion assays (in vitro) were performed.

Insulin secretion assays for the compounds were performed like in paragraph [0208].

Compounds 2, 4, 7, 9, 11, 13, 16, 19, 20, 21, 22, 23, 24, 28, 29, 31, 32, 33, 34, 35, 36, 37, 38, 39, 40, 41 and 42 exhibited a significant effect on insulin secretion from pancreatic islets compared to control.

### Example 6

Further, glucose tolerance tests (in vivo) were performed, if the corresponding in vitro assays were “positive”.

Glucose tolerance tests for the compounds were performed like in paragraph [0209].

Compounds 35 and 36 exhibited a slightly positive effect on blood glucose concentration compared to control.

Compound 7 exhibited a significant effect on blood glucose concentration compared to control.

What is claimed is:

1. A compound according to general formula (I) 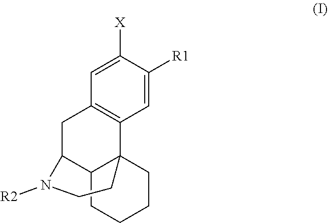
wherein
R1 means —H, —OH, —CO₂H, —R0, —OR0, —OC(═O)R0, —OC(═O)OR0 or —OC(═O)NHR0;
R2 means —H, —R0, —C(═O)R0, —C(═O)OR0 or —C(═O)NHR0;
X means —SR0 or —NR3R4, wherein
R3 means —H, —R0, —C(═O)R0, —C(═O)OR0, —C(═O)NHR0, —C(═O)N(R0)₂, —C(═O)NH₂, —C(═O)CH(NH₂)R0 or —S(═O)₂R0; and R4 means —C(═O)R0, —C(═O)OR0, —C(═O)NHR0, —C(═O)N(R0)₂, —C(═O)NH₂, —C(═O)CH(NH₂)R0 or —S(═O)₂R0; or
R3 and R4 together with the nitrogen atom to which they are attached form an azetidine ring, a pyrrolidine ring, a piperidine ring, a piperazine ring, a pyrazole ring, a triazole ring, an imidazole ring, or a spirocyclic compound containing an azetidine ring and an oxetanyl ring, in each case independently unsubstituted or substituted with one or two substituents independently selected from the group consisting of ═O, —F, —CH₃, —C(═O)NH₂, —C(═O)NHCH₃, —SO₂CH₃, —NH₂ and —N(CH₃)₂;
wherein R0 in each case means -C₁₋₆-alkyl, -C₃₋₁₂-cycloalkyl, -C₁₋₆-alkylene-C₃₋₁₂-cycloalkyl, -C₃₋₁₂-heterocycloalkyl, -C₁₋₆-alkylene-C₃₋₁₂-heterocycloalkyl, -C₆₋₁₄-aryl, -C₁₋₆-alkylene-C₃-6-aryl, -C₃₋₁₄-heteroaryl or —C₁₋₆-alkylene-C₃₋₁₄-heteroaryl, in each case independently unsubstituted or substituted;
wherein in each case -C₁₋₆-alkyl may be linear or branched, saturated or unsaturated, unsubstituted or substituted;
wherein in each case -C₁₋₆-alkylene- may be linear or branched, saturated or unsaturated, unsubstituted or substituted;
wherein in each case -C₃₋₁₂-cycloalkyl may be saturated or unsaturated, unsubstituted or substituted;
wherein in each case -C₃₋₁₂-heterocycloalkyl may be may be saturated or unsaturated, unsubstituted or substituted;
wherein in each case -C₆₋₁₄-aryl may be unsubstituted or substituted;
wherein in each case -C₃₋₁₄-heteroaryl may be unsubstituted or substituted;
wherein in each case “substituted” means mono- or polysubstituted with one or more substituents independently of one another selected from -C₁₋₆-alkyl, —F, —Cl, —Br, —I, and —NH₂;
or a physiologically acceptable salt thereof.

2. The compound according to claim 1, wherein
“-C₃₋₁₂-cycloalkyl” is a non-aromatic, monocyclic, bicyclic or tricyclic moiety comprising 3 to 12 ring carbon atoms but no heteroatoms in the ring; and/or
“-C₃₋₁₂-heterocycloalkyl” is a non-aromatic, monocyclic, bicyclic or tricyclic moiety comprising 3 to 12 ring atoms, wherein each cycle comprises independently of one another 1, 2, 3, 4 or more heteroatoms independently of one another selected from the group consisting of nitrogen, oxygen and sulfur, whereas sulfur may be oxidized (S(═O) or (S(═O)₂), whereas the remaining ring atoms are carbon atoms, and whereas bicyclic or tricyclic systems may share common heteroatom(s); and/or
“-C₆₋₁₄-aryl” is an aromatic, monocyclic, bicyclic or tricyclic moiety comprising 6 to 14 ring carbon atoms but no heteroatoms in the ring; and/or
“-C₃₋₁₄-heteroaryl” means an aromatic, monocyclic, bicyclic or tricyclic moiety comprising 3 to 14 ring atoms, wherein each cycle comprises independently of one another 1, 2, 3, 4 or more heteroatoms independently of one another selected from the group consisting ofnitrogen, oxygen and sulfur, whereas the remaining ring atoms are carbon atoms, and whereas bicyclic or tricyclic systems may share common heteroatom(s).

3. The compound of claim 1, wherein R1 means —OR0; optionally —OC₁₋₆-alkyl, unsubstituted or substituted; optionally —OCH₃.

4. The compound of claim 1, wherein R2 means —R0; optionally -C₁₋₆-alkyl, unsubstituted or substituted; optionally —CH₃.

5. The compound of claim 1, wherein X means —SR0; optionally —S-C₁₋₆-alkyl, unsubstituted or substituted; optionally —S—CH₃; or —S-C₃₋₁₄-heteroaryl, unsubstituted or substituted.

6. The compound of claim 1, wherein X means —NR3R4 and wherein
(i) R3 means —H, —R0, —C(═O)R0, —C(═O)OR0, or —C(═O)NHR0; and/or
(ii) R4 means —C(═O)R0, —C(═O)OR0, —C(═O)NHR0, —C(═O)N(R0)₂, —C(═O)NH₂, —C(═O)CH(NH₂)R0, or —S(═O)₂R0;
or
R3 and R4 together with the nitrogen atom to which they are attached form
an azetidine ring, which is monosubstituted with —N(CH₃)₂ or —SO₂CH₃, or disubstituted with —F;
a pyrrolidine ring, which is monosubstituted with —N(CH₃)₂ or —NH₂;
a piperidine ring, which is unsubstituted or monosubstituted with —C(═O)NHCH₃ or —NH₂;
a piperazine ring, which is disubstituted with ═O and —CH₃;
a pyrazole ring, which is unsubstituted;
a triazole ring, which is unsubstituted;
an imidazole ring, which is monosubstituted with —C(═O)NH₂; or
a spirocyclic compound consisting of an azetidine ring and an oxetanyl ring, which is unsubstituted.

7. The compound of claim 1, wherein R3 means —H, —R0, or —C(═O)NHR0; optionally —H, -C₁₋₆-alkyl, unsubstituted or substituted, or —C(═O)NH—C₁₋₆-alkyl, unsubstituted or substituted; optionally —H, —CH₃, or —C(═O)NH—CH₃.

8. The compound of claim 1, wherein R4 means —R0, —C(═O)R0,
—C(═O)N(R0)₂, —C(═O)NH₂, —C(═O)CH(NH₂)R0 or —S(═O)₂R0; optionally —C(═O)-C₁₋₆-alkyl,
—C(═O)-C₃₋₁₂-cycloalkyl, —C(═O)-C₃₋₁₂-heterocycloalkyl, —C(═O)-C₃₋₁₄-heteroaryl, —C(═O)N(-C₁₋₆-alkyl)₂, —C(═O)NH₂, —C(═O)C(NH₂)-C₃₋₁₂-cycloalkyl or —S(═O)₂—C₁₋₆-alkyl, in each case independently unsubstituted or substituted;
optionally R4 means
(i) —C(═O)-C₃₋₁₂-cycloalkyl, unsubstituted or substituted, or —C(═O)-C₃₋₁₂-heterocycloalkyl, unsubstituted or substituted: optionally a moiety according to general formula (A), (B) or (C) 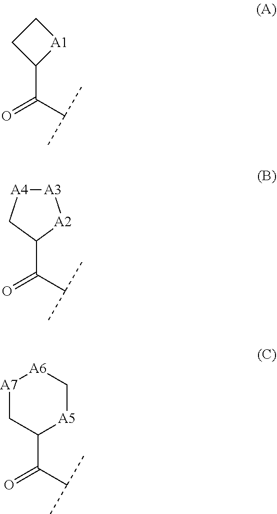
wherein A1, A2, A3, A4, A5, A6 and A7 independently of one another mean —CH₂—, —CHF—, —NH—, —N(CH₃)—, or —O—; or
(ii) —C(═O)-C₃₋₁₄-heteroaryl, unsubstituted or substituted; optionally a moiety according to general formula (D) or (E) 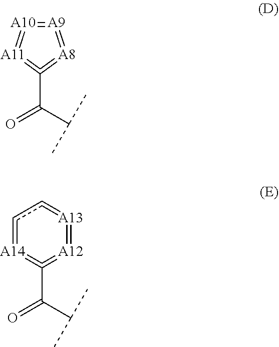
wherein A8, A9, A10, A11, A12, A13 and A14 independently of one another mean —CH═, —C(CH₃)═, —C(NH₂)═, —N═, —NH—, —N(CH₃)—, or —O—;
optionally R4 means —C(═O)CH₃; —C(═O)C(CH₃)₃; —C(═O)—CH₂—CF₃; —C(═O)N(CH₃)₂; —C(═O)NH₂; —C(═O)CH(NH₂)-cyclopropyl; —S(═O)₂—CH₃; or
—C(═O)-C₃₋₁₂-cycloalkyl according to general formula (A), wherein
A1 means —CH₂—; or
—C(═O)-C₃₋₁₂-cycloalkyl according to general formula (B), wherein
A2, A3, and A4 mean —CH₂—; or
—C(═O)-C₃₋₁₂-heterocycloalkyl according to general formula (A), wherein
A1 means —NH—; or
—C(═O)-C₃₋₁₂-heterocycloalkyl according to general formula (B), wherein
A2 means —NH—, and A3 and A4 mean —CH₂—;
A2 means —N(CH₃)—, and A3 and A4 mean —CH₂—;
A2 means —O—, and A3 and A4 mean —CH₂—;
A2 means —NH—, A3 means —CH₂—, and R4 means —CHF—; or
A2 and A4 mean —CH₂—, and A3 means —NH—; or
—C(═O)-C₃₋₁₂-heterocycloalkyl according to general formula (C), wherein
A5 means —O—, A6 means —CH₂—, and A7 means —NH—;
A5 and A7 mean —CH₂—, and A6 means —NH—;
A5 and A7 mean —CH₂—, and A6 means —O—; or
A5 means —NH—, A6 means —CH₂—, and A7 means —N(CH₃)—; or
—C(═O)-C₃₋₁₄-heteroaryl according to general formula (D), wherein
A8 and A11 mean —C(CH₃)═, A9 means —N═, and A10 means —O—;
A8 means —N(CH₃)—, A9 and A11 mean —CH═, and A10 means —N═;
A8 means —N═, A9 and A11 mean —CH═, and A10 means —N(CH₃)—; or
A8 means —N═, A9 and A11 means —CH═, and A10 means —NH—; or
—C(═O)-C₃₋₁₄-heteroaryl according to general formula (E), wherein
A12 means —CH═, A13 and A14 mean —N═; or
A12 means —C(NH₂)═, and A13 and A14 mean —N═.

9. The compound according to claim 8, wherein
(i) X means —NHR4, wherein R4 means
—C(═O)-C₃₋₁₂-heterocycloalkyl according to general formula (B), wherein A2 means —NH—, and A3 and A4 mean —CH₂—;
—C(═O)-C₃₋₁₂-cycloalkyl according to general formula (B), wherein A2, A3, and A4 mean —CH₂—;
—C(═O)-C₃₋₁₄-heteroaryl according to general formula (D), wherein A8 means —N═, A9 and A11 means —CH═, and A10 means —NH—;
—C(═O)-C₃₋₁₂-heterocycloalkyl according to general formula (A), wherein A1 means —NH—;
—C(═O)-C₃₋₁₂-heterocycloalkyl according to general formula (B), wherein A2 and A4 mean —CH₂—, and A3 means —NH—;
—C(═O)-C₃₋₁₂-heterocycloalkyl according to general formula (B), wherein A2 means —O—, and A3 and A4 mean —CH₂—;
—C(═O)-C₃₋₁₄-heteroaryl according to general formula (D), wherein A8 means —N(CH₃)—, A9 and A11 mean —CH═, and A10 means —N═;
—C(═O)-C₃₋₁₄-heteroaryl according to general formula (D), wherein A8 means —N═, A9 and A11 mean —CH═, and A10 means —N(CH₃)—;
—C(═O)-C₃₋₁₂-heterocycloalkyl according to general formula (C), wherein A5 and A7 mean —CH₂—, and A6 means —O—;
—C(═O)-C₃₋₁₂-heterocycloalkyl according to general formula (B), wherein A2 means —NH—, A3 means —CH₂—, and R4 means —CHF—;
—C(═O)-C₃₋₁₂-heterocycloalkyl according to general formula (B), wherein A2 means —N(CH₃)—, and A3 and A4 mean —CH₂—;
—C(═O)-C₃₋₁₄-heteroaryl according to general formula (D), wherein A8 and A11 mean —C(CH₃)═, A9 means —N═, and A10 means —O—;
—C(═O)-C₃₋₁₂-cycloalkyl according to general formula (A), wherein A1 means —CH₂—;
—C(═O)-C₃₋₁₂-heterocycloalkyl according to general formula (C), wherein A5 means —O—, A6 means —CH₂—, and A7 means —NH—;
—C(═O)-C₃₋₁₂-heterocycloalkyl according to general formula (C), wherein A5 and A7 mean —CH₂—, and A6 means —NH—;
—C(═O)—CH₂—CF₃;
—C(═O) C(CH₃)₃;
—C(═O)CH(NH₂)-cyclopropyl;
—C(═O)-C₃₋₁₄-heteroaryl according to general formula (E), wherein A12 means —CH═, A13 and A14 mean —N═;
—C(═O)-C₃₋₁₄-heteroaryl according to general formula (E), wherein A12 means —C(NH₂)═, and A13 and A14 mean —N═;
—C(═O)-C₃₋₁₂-heterocycloalkyl according to general formula (C), wherein A5 means —NH—, A6 means —CH₂—, and A7 means —N(CH₃)—; or
—S(═O)₂—CH₃; or
(ii) X means —N(CH₃)R4, wherein R4 means
—C(═O)CH₃;
—C(═O)NH₂;
—C(═O)NH(CH₃);
—C(═O)N(CH₃)₂; or
—S(═O)₂—CH₃.

10. The compound of claim 1, wherein the compound has a stereochemistry according to general formula (II) 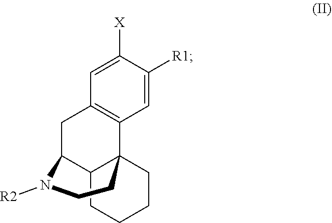
and wherein X, R1 and R2 are defined according to any of the preceding claims.

11. The compound of claim 1, wherein
X does not mean —SCF₃, —SC₆H₅, or —N(H)(C(═O)CH₃); and
R2 does not mean —H or —C(═O)—O—CH₂—C₆H₅.

12. The compound of claim 1, wherein
(i) X is defined according to claim 9; or
(ii) X means —S-C₁₋₆-alkyl, unsubstituted, optionally —S—CH₃; and R2 means -C₁₋₆-alkyl, unsubstituted, optionally —CH₃.

13. The compound of claim 12, wherein in embodiment (i) R2 means -C₁₋₆-alkyl, unsubstituted, optionally —CH₃.

14. The compound of claim 12, wherein in embodiments (i) and (ii) R1 means —OC₁₋₆-alkyl, unsubstituted, optionally —OCH₃.

15. The compound of claim 1, wherein the compound is selected from a group consisting of
(2R)-N-[(1S,9S)-4-methoxy-17-methyl-17-azatetracyclo[7.5.3.0¹,¹⁰,0²,⁷]heptadeca-2(7),3,5-trien-5-yl]pyrrolidine-2-carboxamide;
N-[(1S,9S)-4-methoxy-17-methyl-17-azatetracyclo[7.5.3.0¹,¹⁰,0²,⁷]heptadeca-2(7),3,5-trien-5-yl]cyclopentanecarboxamide;
N-[(1S,9S)-4-methoxy-17-methyl-17-azatetracyclo[7.5.3.0¹,¹⁰,0²,⁷]heptadeca-2(7),3,5-trien-5-yl]-1H-imidazole-4-carboxamide;
N-[(1S,9S)-4-methoxy-17-methyl-17-azatetracyclo[7.5.3.0¹,¹⁰,0²,⁷]heptadeca-2(7),3,5-trien-5-yl]azetidine-2-carboxamide;
N-[(1S,9S)-4-methoxy-17-methyl-17-azatetracyclo[7.5.3.0¹,¹⁰,0²,⁷]heptadeca-2(7),3,5-trien-5-yl]pyrrolidine-3-carboxamide;
N-[(1S,9S)-4-methoxy-17-methyl-17-azatetracyclo[7.5.3.0¹,¹⁰,0²,⁷]heptadeca-2(7),3,5-trien-5-yl]oxolane-2-carboxamide;
N-[(1S,9S)-4-methoxy-17-methyl-17-azatetracyclo[7.5.3.0¹,¹⁰,0²,⁷]heptadeca-2(7),3,5-trien-5-yl]-1-methyl-1H-imidazole-5-carboxamide; dihydrochloride;
N-[(1S,9S)-4-methoxy-17-methyl-17-azatetracyclo[7.5.3.0¹,¹⁰,0²,⁷]heptadeca-2(7),3,5-trien-5-yl]-1-methyl-1H-imidazole-4-carboxamide;
N-[(1S,9S)-4-methoxy-17-methyl-17-azatetracyclo[7.5.3.0¹,¹⁰,0²,⁷]heptadeca-2(7),3,5-trien-5-yl]oxane-4-carboxamide;
4-fluoro-N-[(1S,9S)-4-methoxy-17-methyl-17-azatetracyclo[7.5.3.0¹,¹⁰,0²,⁷]heptadeca-2(7),3,5-trien-5-yl]pyrrolidine-2-carboxamide;
N-[(1S,9S)-4-methoxy-17-methyl-17-azatetracyclo[7.5.3.0¹,¹⁰,0²,⁷]heptadeca-2(7),3,5-trien-5-yl]-1-methylpyrrolidine-2-carboxamide;
N-[(1S,9S)-4-methoxy-17-methyl-17-azatetracyclo[7.5.3.0¹,¹⁰,0²,⁷]heptadeca-2(7),3,5-trien-5-yl]-3,5-dimethyl-1,2-oxazole-4-carboxamide;
N-[(1S,9S)-4-methoxy-17-methyl-17-azatetracyclo[7.5.3.0¹,¹⁰,0²,⁷]heptadeca-2(7),3,5-trien-5-yl]cyclobutanecarboxamide;
N-[(1S,9S)-4-methoxy-17-methyl-17-azatetracyclo[7.5.3.0¹,¹⁰,0²,⁷]heptadeca-2(7),3,5-trien-5-yl]morpholine-2-carboxamide;
N-[(1S,9S)-4-methoxy-17-methyl-17-azatetracyclo[7.5.3.0¹,¹⁰,0²,⁷]heptadeca-2(7),3,5-trien-5-yl]piperidine-4-carboxam;
3,3,3-trifluoro-N-[(1S,9S)-4-methoxy-17-methyl-17-azatetracyclo[7.5.3.0¹,¹⁰,0²,⁷]heptadeca2(7),3,5-trien-5-yl]propanamide;
N-[(1S,9S)-4-methoxy-17-methyl-17-azatetracyclo[7.5.3.0¹,¹⁰,0²,⁷]heptadeca-2(7),3,5-trien-5-yl]-2,2-dimethylpropanamide;
(2S)-2-amino-2-cyclopropyl-N-[(1S,9S)-4-methoxy-17-methyl-17-azatetracyclo[7.5.3.0¹,¹⁰,0²,⁷]heptadeca-2(7),3,5-trien-5-yl]acetamide;
N-[(1S,9S)-4-methoxy-17-methyl-17-azatetracyclo[7.5.3.0¹,¹⁰,0²,⁷]heptadeca-2(7),3,5-trien-5-yl]pyrazine-2-carboxamide;
3-amino-N-[(1S,9S)-4-methoxy-17-methyl-17-azatetracyclo[7.5.3.0¹,¹⁰,0²,⁷]heptadeca-2(7),3,5-trien-5-yl]pyrazine-2-carboxamide;
N-[(1S,9S)-4-methoxy-17-methyl-17-azatetracyclo[7.5.3.0¹,¹⁰,0²,⁷]heptadeca-2(7),3,5-trien-5-yl]-N-methylacetamide;
1-[(1S,9S)-4-methoxy-17-methyl-17-azatetracyclo[7.5.3.0¹,¹⁰,0²,⁷]heptadeca-2(7),3,5-trien-5-yl]-1-methylurea;
1-[(1S,9S)-4-methoxy-17-methyl-17-azatetracyclo[7.5.3.0¹,¹⁰,0²,⁷]heptadeca-2(7),3,5-trien-5-yl]-1,3-dimethylurea;
1-[(1S,9S)-4-methoxy-17-methyl-17-azatetracyclo[7.5.3.0¹,¹⁰,0²,⁷]heptadeca-2(7),3,5-trien-5-yl]-1,3,3-trimethylurea;
(2S)—N-[(1S,9S)-4-methoxy-17-methyl-17-azatetracyclo[7.5.3.0¹,¹⁰,0²,⁷]heptadeca-2(7),3,5-trien-5-yl]pyrrolidine-2-carboxamide;
N-[(1S,9S)-4-methoxy-17-methyl-17-azatetracyclo[7.5.3.0¹,¹⁰,0²,⁷]heptadeca-2(7),3,5-trien-5-yl]-4-methylpiperazine-2-carboxamide;
N-[(1S,9S)-4-methoxy-17-methyl-17-azatetracyclo[7.5.3.0¹,¹⁰,0²,⁷]heptadeca-2(7),3,5-trien-5-yl]methanesulfonamide;
N-[(1S,9S)-4-methoxy-17-methyl-17-azatetracyclo[7.5.3.0¹,¹⁰,0²,⁷]heptadeca-2(7),3,5-trien-5-yl]methanesulfonamide;
(1S,9S)-4-methoxy-17-methyl-5-(methylsulfanyl)-17-azatetracyclo[7.5.3.0¹,¹⁰,0²,⁷]heptadeca2(7),3,5-triene;
1-[(1S,9S)-4-methoxy-17-methyl-17-azatetracyclo[7.5.3.0¹,¹⁰,0²,⁷]heptadeca-2,4,6-trien-5-yl]-1H-imidazole-4-carboxamide;
1-[(1S,9S)-4-methoxy-17-methyl-17-azatetracyclo[7.5.3.0¹,¹⁰,0²,⁷]heptadeca-2(7),3,5-trien-5-yl]-N,N-dimethylpyrrolidin-3-amine;
1-[(1S,9S)-4-methoxy-17-methyl-17-azatetracyclo[7.5.3.0¹,¹⁰,0²,⁷]heptadeca-2(7),3,5-trien-5-yl]-N-methylpiperidine-3-carboxamide;
1-[(1S,9S)-4-methoxy-17-methyl-17-azatetracyclo[7.5.3.0¹,¹⁰,0²,⁷]heptadeca-2(7),3,5-trien-5-yl]pyrrolidin-3-amine;
1-[(1S,9S)-4-methoxy-17-methyl-17-azatetracyclo[7.5.3.0¹,¹⁰,0²,⁷]heptadeca-2(7),3,5-trien-5-yl]piperidin-3-amine;
1-[(1S,9S)-4-methoxy-17-methyl-17-azatetracyclo[7.5.3.0¹,¹⁰,0²,⁷]heptadeca-2(7),3,5-trien-5-yl]-N,N-dimethylazetidin-3-amine;
4-[(1S,9S)-4-methoxy-17-methyl-17-azatetracyclo[7.5.3.0¹,¹⁰,0²,⁷]heptadeca-2(7),3,5-trien-5-yl]-1-methylpiperazin-2-one;
(1S,9S)-4-methoxy-17-methyl-5-{2-oxa-6-azaspiro[3.3]heptan-6-yl}-17-azatetracyclo[7.5.3.0¹,¹⁰,0²,⁷]heptadeca-2(7),3,5-triene;
(1S,9S)-5-(3,3-difluoroazetidin-1-yl)-4-methoxy-17-methyl-17-azatetracyclo[7.5.3.0¹,¹⁰,0²,⁷]heptadeca-2(7),3,5-triene;
(1S,9S)-4-methoxy-17-methyl-5-(1H-1,2,4-triazol-1-yl)-17-azatetracyclo[7.5.3.0¹,¹⁰,0²,⁷]heptadeca-2,4,6-triene;
(1S,9S)-4-methoxy-17-methyl-5-(1H-pyrazol-1-yl)-17-azatetracyclo[7.5.3.0¹,¹⁰,0²,⁷]heptadeca2,4,6-triene;
(1S,9S)-4-methoxy-17-methyl-5-(piperidin-1-yl)-17-azatetracyclo[7.5.3.0¹,¹⁰,0²,⁷]heptadeca2,4,6-triene; and
(1S,9S)-5-(3-methanesulfonylazetidin-1-yl)-4-methoxy-17-methyl-17-azatetracyclo[7.5.3.0¹,¹⁰,0²,⁷]heptadeca-2(7),3,5-triene;
or a physiologically acceptable salt thereof.

16. A pharmaceutical composition comprising the compound of claim 1.

17. The pharmaceutical composition of claim 16 for use as medicament.

18. A method comprising a step of treatment or prevention of a disease or condition with the pharmaceutical composition of 16 for use in the treatment and/or prevention of diseases or conditions, optionally those involving oxidative stress-induced cell death, selected from
diabetes mellitus, optionally insulin-dependent diabetes mellitus and/or non-insulin-dependent diabetes mellitus and their complications; and/or
obesity, stroke, retinopathy, cardiovascular diseases, such as acute coronary syndrome, myocardial infarction, ischemia-reperfusion injury, diseases of the kidney, such as nephropathy, and diseases of the peripheral nervous system, such as neuropathy.

19. The method of claim 18, wherein the pharmaceutical composition is
administered orally, intravenously, intramuscularly, intrathecally, subcutaneously, sublingually, buccally, rectally, vaginally, ocularly, nasally, and/or cutaneously; and/or
administered once daily, twice daily, thrice daily, or four times per day; or administered once weekly, twice weekly, thrice weekly, or four times per week.

20. The compound of claim 1, wherein the compound is a medicament,
optionally for the treatment and/or prevention of one or more diseases or one or more conditions selected from
diabetes mellitus, optionally insulin-dependent diabetes mellitus and/or noninsulin-dependent diabetes mellitus and their complications; and/or
obesity, stroke, retinopathy, cardiovascular diseases, such as acute coronary syndrome, myocardial infarction, ischemia-reperfusion injury, diseases of the kidney, such as nephropathy, and diseases of the peripheral nervous system, such as neuropathy;
optionally those involving oxidative stress-induced cell death.
